(19) United States
(12) Patent Application Publication
(10) Pub. No.: US 2024/0307088 A1
(43) Pub. Date: Sep. 19, 2024

(54) SYSTEMS AND METHODS FOR HANDHELD REAL-TIME SURGICAL NAVIGATION GUIDANCE

(71) Applicant: Zeta Surgical Inc, Boston, MA (US)

(72) Inventors: Raahil Mohammed Sha, Allen, TX (US); Avinash Lal, Woodhaven, NY (US); Benjamin Hoyoung Lee, Allston, MA (US); Jose Maria Amich Manero, Boston, MA (US)

(73) Assignee: Zeta Surgical Inc, Boston, MA (US)

(21) Appl. No.: 18/272,384

(22) PCT Filed: Jan. 13, 2022
(86) PCT No.: PCT/US2022/012329

§ 371 Date: Jul. 14, 2023

Related U.S. Application Data
(60) Provisional application No. 63/137,455, filed on Jan. 14, 2021.

(51) Int. Cl. A61B 17/34; A61B 34/00; A61B 34/20

(52) U.S. Cl. CPC A61B 17/3403; A61B 34/20; A61B 34/25; A61B 2034/2057; A61B 2034/2068; A61B 2034/252; A61B 2034/258

(57) ABSTRACT

Systems and methods for tracking and aligning a surgical instrument having a configurable display are disclosed. The surgical instrument can include a display that is in communication with one or more computing devices mounted on the surgical instrument. The computing devices mounted on the surgical instrument can communicate with processing circuitry that tracks the position of the surgical instrument in a surgical environment. The processing circuitry can map the position of the surgical instrument within a 3D space co-registered with a medical image of a patient, and can provides prompts for presentation on the display mounted on the surgical device. The prompts can indicate instructions for a surgeon to position the surgical device at a target location in a patient.

### CROSS-REFERENCE TO RELATED APPLICATIONS

The present application claims the benefit of and priority to U.S. Provisional Patent Application No. 63/137,455, filed Jan. 14, 2021, entitled “SYSTEMS AND METHODS FOR HANDHELD REAL-TIME SURGICAL NAVIGATION GUIDANCE,” the contents of which is incorporated herein by reference in its entirety for all purposes.

### BACKGROUND

Positioning surgical tools within a patient can be challenging. For example, surgeons must often alternate between different sources of information during a surgical procedure to achieve desired results.

### SUMMARY

The present disclosure relates generally to the field of instrument tracking and hand-held displays. More particularly, the present disclosure describes a tool, such as a surgical tool, with a coupled display that provides instructions to a surgeon about position the tool within a patient during a surgical procedure. The tool allows a surgeon to access the guidance prompts during a surgical procedure without away from the patient.

At least one aspect of the present disclosure is directed to a device. The device can include a tool portion configured to be inserted into a target location in a patient during a procedure. The device can include a display assembly mounted on the device. The display assembly can include a display. The display can be configured to display prompts for an operator of the device to guide the tool portion to the target location in the patient. The device can include a computing device having one or more processors coupled to memory. The device can provide tool information to a controller computing device. The device can receive, from the controller computing device, instructions to present a change in a position of the device to bring the tool portion closer to the target location in the patient. The device can present, based on the instructions, a guidance prompt for the operator that indicates the change in the position of the device.

In some implementations, the device further includes a grip portion that allows the operator to hold and position the device. In some implementations, the device further includes a housing that houses both the display assembly and the computing device. In some implementations, the tool portion is coupled to the housing. In some implementations, the device further includes a button that, when actuated, causes the tool portion to perform a function of the tool portion. In some implementations, the device further includes one or more position sensors, and the device can receive the tool information from the one or more position sensors.

In some implementations, the device further includes one or more indicators, where each of the one or more indicators mounted at a respective predetermined position on the device. In some implementations, the tool portion is one of a catheter device, a drill device, a biopsy needle, or a cannula needle. In some implementations, the device further includes a respective power interface for each of the computing device and the tool portion. In some implementations, the device further includes a wireless interface, and wherein the computing device configured to provide the tool information and receive the instructions via the wireless interface.

At least one other aspect of the present disclosure is directed to a system. The system can include a connector configured to couple to a body of a surgical tool. The system can include a display assembly coupled to the connector. The display assembly can include a display. The display can display prompts for an operator of the surgical tool to guide the surgical tool to a target location in a patient. The system can include a computing device coupled to the display assembly or the connector. The system can provide tool information about the surgical tool to a controller computing device. The system can receive, from the controller computing device, instructions to present a change in a position of the surgical tool to bring the surgical tool closer to the target location in the patient. The system can present, based on the instructions, a guidance prompt for the operator that indicates the change in the position of the surgical tool.

In some implementations, the system can include the surgical tool, where the surgical tool further includes a grip portion that allows the operator to hold and position the surgical tool while the connector is coupled to the body of the surgical tool. In some implementations, the connector includes a clamp that couples to the body of the surgical tool. In some implementations, the connector is a bracket, and the display assembly or the computing device is coupled to the bracket using threaded screws or bolts. In some implementations, the system can include power distribution circuitry that provides power to the display assembly and the computing device.

In some implementations, the system can include one or more position sensors, and the system can receive the tool information from the one or more position sensors. In some implementations, the system can include one or more indicators, where each of the one or more indicators mounted at a respective predetermined position on the system. In some implementations, the system can include a communications interface via which the computing device communicates data with the controller computing device. In some implementations, the communications interface attaches to a power interface of the surgical tool to receive power for the computing device and the display assembly. In some implementations, the system includes a wireless interface, and the system can provide the tool information and receive the instructions via the wireless interface.

At least one other aspect of the present disclosure is directed to a method. The method can include identifying tool information from a tool having a mounted display assembly coupled to a computing device. The method can include tracking, using signals received from an image capture device, a position of the tool based on determined positions of indicators mounted on the tool. The method can include determining a position of the tool in a three-dimensional (3D) reference frame that includes a target location in a patient. The method can include determining a change in the position of the tool that causes a portion of the tool to move closer to the target location in the 3D reference frame. The method can include generating, based on the change in the position of the tool determined by the one or more processors, display instructions that cause the tool to display a prompt to a user of the tool to adjust the position of the tool. The method can include providing the display instructions to the computing device mounted on the tool.

In some implementations, identifying the tool information from the tool comprises receiving an indication of a type of the tool. In some implementations, the method can include retrieving a 3D medical image of the patient comprising the target location. In some implementations, tracking the position of the tool further comprises performing a calibration procedure for the tool. In some implementations, the calibration procedure comprises mapping the determined positions of the indicators mounted on the tool to the 3D reference frame. In some implementations, determining the position of the tool in the 3D reference frame is further based on a relative distance between a tool end of the tool and the determined positions of the indicators mounted on the tool.

In some implementations, determining the change in the position of the tool further comprises determining a distance between the tool and the target location. In some implementations, determining the change in the position of the tool is further based on sensor data received from one or more sensors mounted on the tool. In some implementations, generating the display instructions further comprises transforming the distance between the tool and the target location to a reference frame of the mounted display assembly. In some implementations, the display instructions comprise instructions to display one or more indicators when the tool is positioned at the target location.

At least one other aspect of the present disclosure is directed to a system. The system can include one or more processors coupled to memory. The system can identify tool information from a tool having a mounted display assembly coupled to a computing device. The system can track, using signals received from an image capture device, a position of the tool based on determined positions of indicators mounted on the tool. The system can determine a position of the tool in a three-dimensional reference frame that includes a target location in a patient. The system can determine a change in the position of the tool that causes a portion of the tool to move closer to the target location in the three-dimensional reference frame. The system can generate, based on the change in the position of the tool determined by the one or more processors, display instructions that cause the tool to display a prompt to a user of the tool to adjust the position of the tool. The system can provide the display instructions to the computing device mounted on the tool.

In some implementations, to identify the tool information from the tool, the system can receive an indication of a type of the tool. In some implementations, the system can retrieve a 3D medical image of the patient comprising the target location. In some implementations, to track the position of the tool, the system can perform a calibration procedure for the tool. In some implementations, to perform the calibration procedure, the system can map the determined positions of the indicators mounted on the tool to the 3D reference frame. In some implementations, the system can determine the position of the tool in the 3D reference frame further based on a relative distance between a tool end of the tool and the determined positions of the indicators mounted on the tool.

In some implementations, to determine the change in the position of the tool, the system can determine a distance between the tool and the target location. In some implementations, the system can determine the change in the position of the tool further based on sensor data received from one or more sensors mounted on the tool. In some implementations, to generate the display instructions, the system can transform the distance between the tool and the target location to a reference frame of the mounted display assembly. In some implementations, the display instructions comprise instructions to display one or more indicators when the tool is positioned at the target location.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. Aspects can be combined and it will be readily appreciated that features described in the context of one aspect of the invention can be combined with other aspects. Aspects can be implemented in any convenient form. For example, by appropriate computer programs, which can be carried on appropriate carrier media (computer readable media), which can be tangible carrier media (e.g. disks) or intangible carrier media (e.g. communications signals). Aspects can also be implemented using suitable apparatus, which can take the form of programmable computers running computer programs arranged to implement the aspect. As used in the specification and in the claims, the singular form of ‘a’, ‘an’, and ‘the’ include plural referents unless the context clearly dictates otherwise.

### BRIEF DESCRIPTION OF THE DRAWINGS

The accompanying drawings are not intended to be drawn to scale. Like reference numbers and designations in the various drawings indicate like elements. For purposes of clarity, not every component can be labeled in every drawing. In the drawings:

FIGS. 1A and 1B show perspective views of an example image processing system, in accordance with one or more implementations;

FIG. 2 is a block diagram of an image processing system capable of tracking a position of a tool, in accordance with one or more implementations;

FIGS. 3A and 3B show perspective views an example tool with an integrated display device, in accordance with one or more implementations;

FIGS. 4A and 4B show perspective views of an example tool assembly similar to the tool shown in FIGS. 3A and 3B, in accordance with one or more implementations;

FIG. 5 shows a perspective view of an example tool with a mounting bracket having a computing device and a display assembly, in accordance with one or more implementations;

FIGS. 6A and 6B show perspective views of the tool and the bracket shown in FIG. 5, respectively, in accordance with one or more implementations;

FIG. 7 shows an block diagram of an example system for tracking and providing display instructions to a tool with an integrated display, in accordance with one or more implementations;

FIG. 8 is a flow diagram of an example method of tracking and providing display instructions to a tool with an integrated display, in accordance with one or more implementations;

FIGS. 9A and 9B are block diagrams of an example computing environment, in accordance with one or more implementations; and

FIGS. 10A, 10B, 10C, 10D, 10E, 10F, and 10G show example views of an example tool assembly similar to the devices described herein, in accordance with one or more implementations.

### DETAILED DESCRIPTION

Below are detailed descriptions of various concepts related to, and implementations of, techniques, approaches, methods, apparatuses, and systems for managing surgical tools having integrated display devices. The various concepts introduced above and discussed in greater detail below can be implemented in any of numerous ways, as the described concepts are not limited to any particular manner of implementation. Examples of specific implementations and applications are provided primarily for illustrative purposes.

For purposes of reading the description of the various embodiments below, the following descriptions of the sections of the Specification and their respective contents can be helpful:

Section A describes techniques for tracking the position of a surgical tool in a surgical environment and presenting movement prompts on a display mounted to the surgical tool; and

Section B describes a computing environment which can be useful for practicing implementations described herein.

### A. Tracking a Position of Surgical Tool Having an Integrated Display, and Presenting Movement Instructions Thereon

The systems and methods described describe a tool, such as a surgical tool, which can include a display device that presents instructions to a user, such as a surgeon or other medical professional, to aid in a procedure. The display can form part of the tool, or can be mounted to the tool using a bracket. The bracket, or the tool itself, can include a computing device that can present information on the display. The computing device can be in communication with a main computing system that tracks the tool in a surgical environment, for example, during a procedure. The tool described herein provides benefits to surgeons and other medical professionals by providing real-time prompts to guide the tool to a target location within a patient. By mounting the display onto the surgical tool, the surgeon or medical professional does not need to look away from the portion of the patient being operated upon. Systems and methods in accordance with the present disclosure can selectively, accurately, and at appropriate times during procedures present information to the user to enable more effective situational awareness for the user and performance of the procedure. For example, the systems and methods of the present disclosure can evaluate position information from a surgical tool to accurately present information to aid in positioning the surgical tool (e.g., move left, move down, etc.) at appropriate times during a surgical procedure. Unlike other display technologies, the systems and methods described herein include a small and power efficient display mounted directly on a surgical tool, allowing the user to view the presented information without looking away from the procedure being performed.

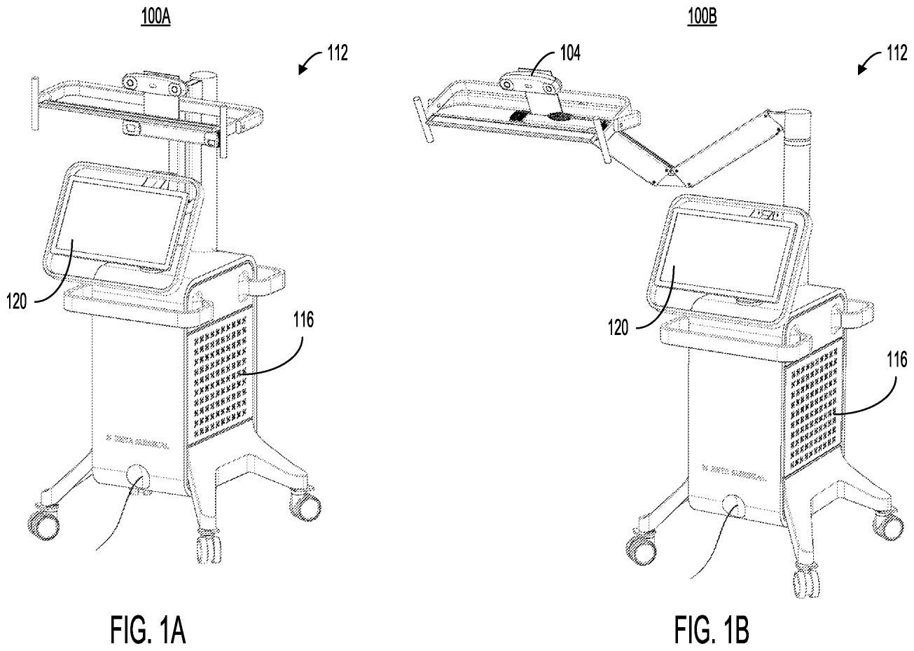

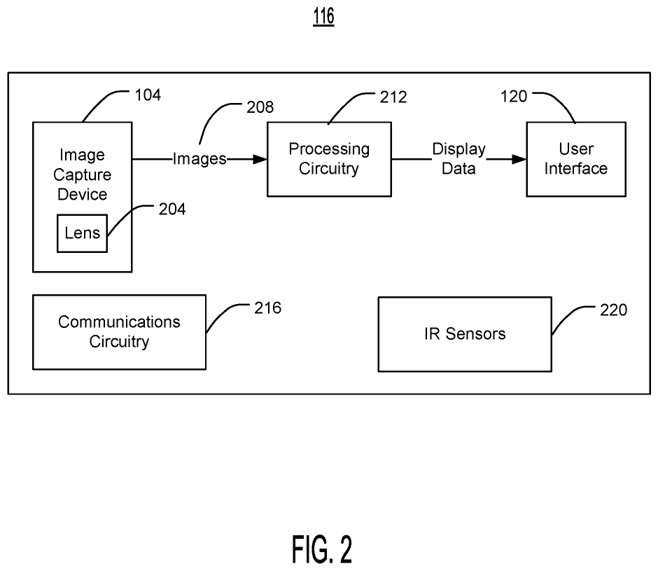

FIGS. 1A, 1B and 2 depict an image processing system 100. The image processing system 100 can include a plurality of image capture devices 104, such as three-dimensional cameras. The cameras can be visible light cameras (e.g., color or black and white), infrared cameras (e.g., the IR sensors 220, etc.), or combinations thereof. Each image capture device 104 can include one or more lenses 204. In some embodiments, the image capture device 104 can include a camera for each lens 204. The image capture devices 104 can be selected or designed to be a predetermined resolution and/or have a predetermined field of view. The image capture devices 104 can have a resolution and field of view for detecting and tracking objects. The image capture devices 104 can have pan, tilt, or zoom mechanisms. The image capture device 104 can have a pose corresponding to a position and orientation of the image capture device 104. The image capture device 104 can be a depth camera. The image capture device 104 can be the KINECT manufactured by MICROSOFT CORPORATION.

Light of an image to be captured by the image capture device 104 be received through the one or more lenses 204. The image capture devices 104 can include sensor circuitry, including but not limited to charge-coupled device (CCD) or complementary metal-oxide-semiconductor (CMOS) circuitry, which can detect the light received via the one or more lenses 204 and generate images 208 based on the received light.

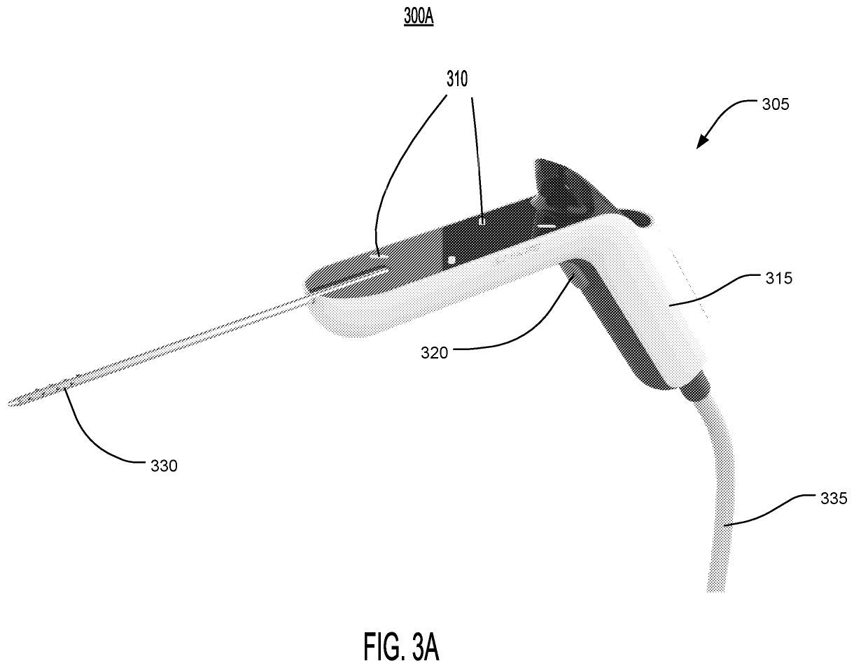

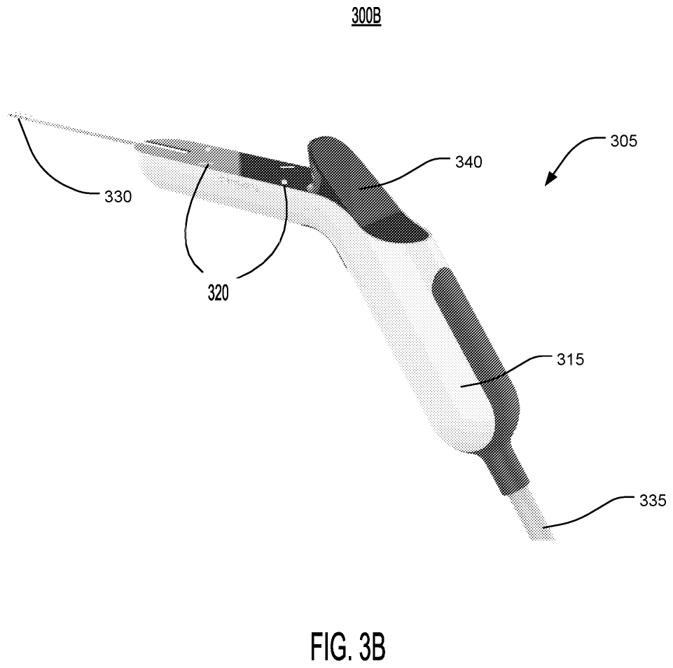

The image capture devices 104 can provide images 208 to processing circuitry 212, for example via a communications bus. The image capture devices 104 can provide the images 208 with a corresponding timestamp, which can facilitate synchronization of the images 208 when image processing is executed on the images 208. The image capture devices 104 can output 3D images (e.g., images having depth information). The images 208 can include a plurality of pixels, each pixel assigned spatial position data (e.g., horizontal, vertical, and depth data), intensity or brightness data, and/or color data. When captured in a surgical environment that includes a tool with mounted indicators, such as the tools 305, 405, or 505 described herein below in conjunction with FIGS. 3A-5, the images 208 can include pixels that represent the indicators (e.g., the indicators 310, 410, or 510, etc.) mounted on the tools in the surgical environment. If the image capture devices 104 are 3D cameras, each of the indicators on the tools present in the surgical environment can be associated with a 3D point in the reference frame of the image capture devices 104.

Each image capture device 104 can be coupled with the platform 112, such as via one or more arms or other supporting structures, and can be communicatively coupled to the processing circuitry 212. The platform 112 can be a cart that can include wheels for movement and various support surfaces for supporting devices to be used with the platform 112. In some implementations, the platform is a fixed structure without wheels, such as a table. In some implementations, the components coupled to the platform 112 can be modular and removable, such that they can be replaced with other tracking devices or computing devices as needs arise.

The platform 112 can support processing hardware 116 (which is described in further detail below in conjunction with FIG. 2) that includes at least a portion of processing circuitry 212, as well as user interface 120. The user interface 120 can be any kind of display or screen as described herein, and can be used to display a three-dimensional rendering of the environment captured by the image capture devices 104. Images 208 can be processed by processing circuitry 212 for presentation via user interface 120. As described above, the images 208 can include indications of a location of indicators present on tool devices positioned within the three-dimensional environment captured by the image capture devices 104. In some implementations, the processing circuitry 212 can utilize one or more image classification techniques (e.g., deep neural networks, light detection, color detection, etc.) to determine the location (e.g., pixel location, 3D point location, etc.) of indicators mounted to tool devices in the three-dimensional environment.

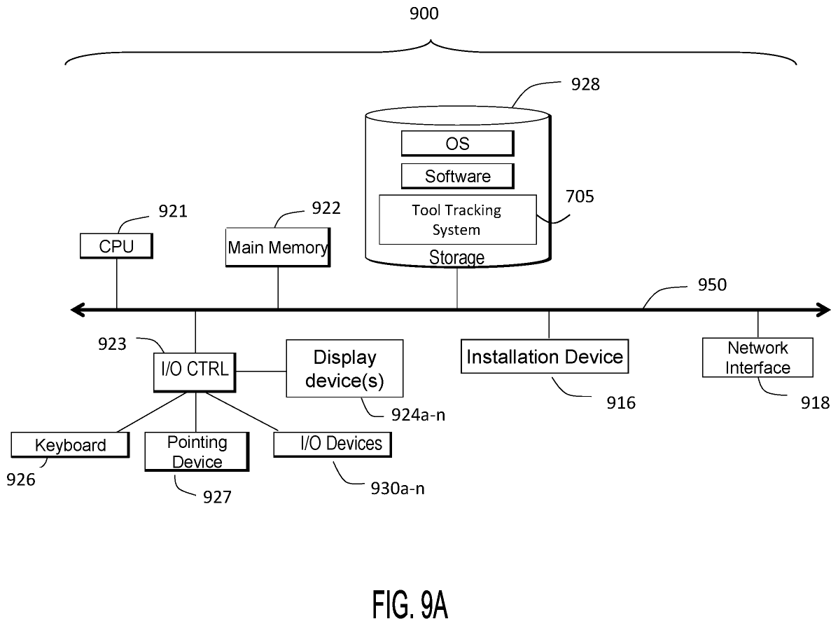

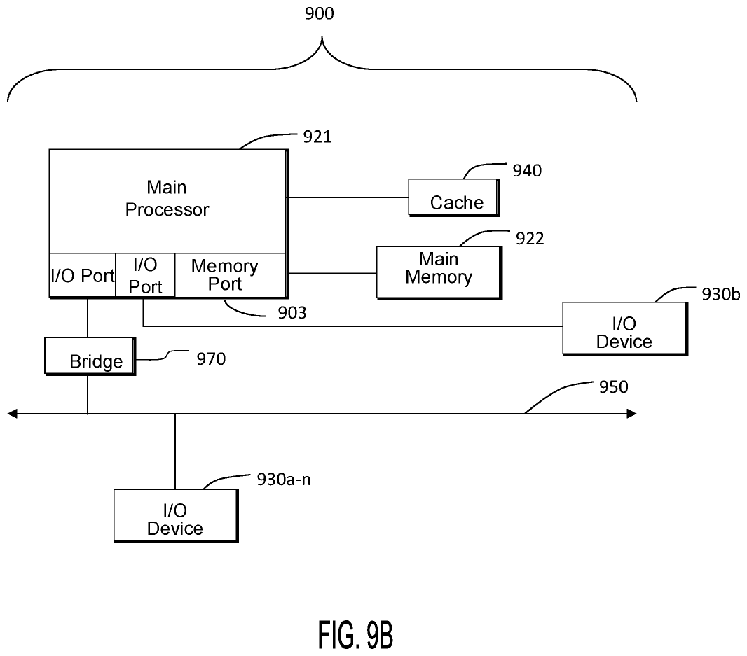

Processing circuitry 212 can incorporate features of computing device 900 described with reference to FIGS. 9A and 9B. For example, processing circuitry 212 can include processor(s) and memory. The processor can be implemented as a specific purpose processor, an application specific integrated circuit (ASIC), one or more field programmable gate arrays (FPGAs), a group of processing components, or other suitable electronic processing components. The memory is one or more devices (e.g., RAM, ROM, flash memory, hard disk storage) for storing data and computer code for completing and facilitating the various user or client processes, layers, and modules described in the present disclosure. The memory can be or include volatile memory or non-volatile memory and can include database components, object code components, script components, or any other type of information structure for supporting the various activities and information structures of the inventive concepts disclosed herein. The memory is communicably connected to the processor and includes computer code or instruction modules for executing one or more processes described herein. The memory includes various circuits, software engines, and/or modules that cause the processor to execute the systems and methods described herein.

Some portions of processing circuitry 212 can be provided by one or more devices remote from platform 112. For example, one or more servers, cloud computing systems, or mobile devices (e.g., as described with reference to FIGS. 9A and 9B), can be used to perform various portions of the image processing pipeline described herein.

The image processing system 100 can include communications circuitry 216. The communications circuitry 216 can implement features of computing device 900 described with reference to FIGS. 9A and 9B, such as network interface 918. The communications circuitry 216 can be used, for example, to communicate instructions to a surgical tool, such as the tool 305, 405, or 505 depicted in FIGS. 3A-5. In some implementations, the communications circuitry 216 can be used to receive sensor information from the tools 305, 405, and 505, such as gyroscope or accelerometer information, which can be used by the processing circuitry 212 to determine an adjusted position for the tool.

The image processing system 100 can include one or more infrared (IR) sensors 220. The IR sensors 220 can detect IR signals from various devices in an environment around the image processing system 100. For example, the IR sensors 220 can be used to detect IR signals from IR emitters that can be coupled with instruments (e.g., the tools 305, 405, or 505, etc.) in order to track the instruments. The IR sensors 220 can be communicatively coupled to the other components of the image processing system 100, such that the components of the image processing system 100 can utilize the IR signals in appropriate operations in the image processing pipeline, as described herein below.

Referring now to FIGS. 3A and 3B, depicted are respective perspective views 300A and 300B of an example surgical tool 305 that can be used in conjunction with the image processing devices (e.g., the image processing system 100, the tool tracking system 705, etc.) described herein. The tool 305 can be an adapted version of an existing surgical tool that includes additional tracking features and a display that can aid a surgeon in positioning the tool 305 at a target location, for example within a patient. The tool 305 can be, for example, a catheter device, a drill device, a biopsy needle, or a cannula needle, among others.

The tool 305 can include tracking indicators 310. The tracking indicators can be, for example, IR light-emitting diodes (LEDs), LEDs that emit color in the visual spectrum, tracking balls colored with a predetermined color or having a predetermined, detectable shape, or other tracking features, such as QR codes. The tracking indicators 310 can be positioned on predetermined places on the tool 305, and can form a matrix or array of sensors that, when detected by a computing device (e.g., the image processing system 100, the tool tracking system 705, etc.), can be used to determine a position and orientation of the tool 305. In some implementations, the tool 305 can include one or more position sensors, such as accelerometers, gyroscopes, or inertial measurement units (IMUs), among others.

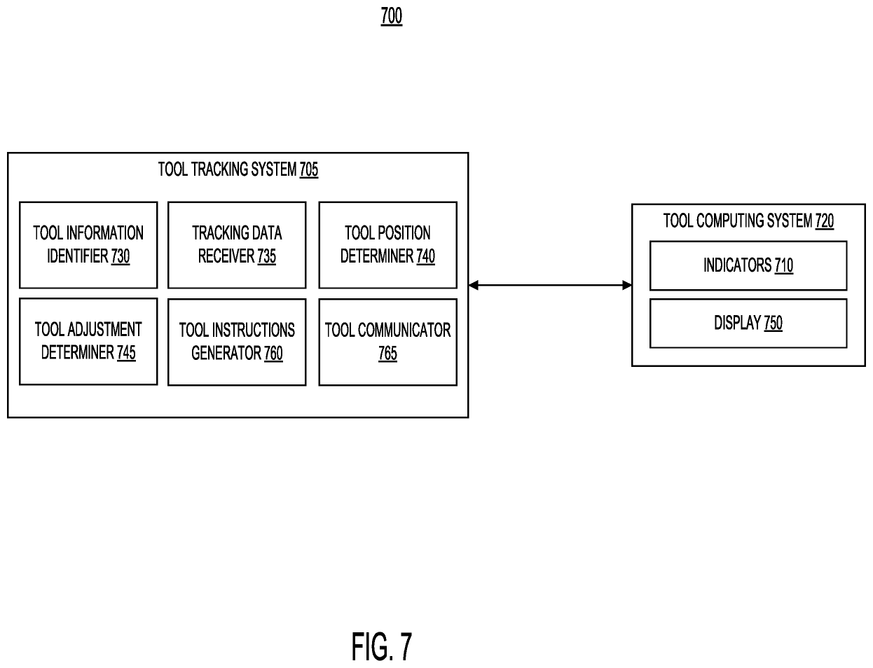

The tool 305 can include a grip portion 315 that a surgeon, or in some implementations, another surgical robot, can use to hold or attach to the tool. As depicted in FIGS. 3A and 3B, this is depicted as a handle. The grip portion can include grooves, a rubber sheathe, or a surface that can facilitate holding the device for long periods of time. In some implementations, the tool 305 can include a computing device, such as the tool computing device 720 described herein below in conjunction with FIG. 7, in the grip portion 315. However, it should be understood that a computing device can be included anywhere within, or coupled to the surface of, the tool 305. The computing device coupled to the tool 305 can be communicatively coupled with other devices mounted on the tool, such as the indicators 310 and the display 340.

The tool 305 can include a button that causes the tool to perform its designed function. A drill, for example, can rotate the tool end (e.g., the tool end 330) in response to a surgeon pressing the button 320. In some implementations, the button 320 can be used to provide input signals to the computing device coupled to the tool 305. For example, the button 320 can be used to switch between target positions within the patient, switch between configuration settings as described herein, provide input to a tool tracking system (e.g., the tool tracking system 705, etc.), or navigate one or more user interfaces displayed on the display 340, among other functionalities. The button 320 can be a toggle button (e.g., active when pressed, and deactivated when pressed again, etc.), or can be activated in response to a pressing or releasing the button 320. The button 320 can be communicatively coupled with the computing device positioned on (or within) the tool 305, and can provide one or more signals to the computing device to carry out one or more functionalities described herein.

The tool 305 can include a tool end 330, which can be positioned within the patient. In general, the grip portion 315 of the tool 305 held by a surgeon, robot, or other medical professional is positioned outside of a patient throughout a medical procedure. The tool end 330, which can have a tip portion, can be positioned by the surgeon at a target position within the patient to carry out a portion of a medical procedure, such as biopsy. Information about the tool end 330, such as the dimensions of the tool end, the type of tool 305, the distance and orientation of the tool end 330 from the tracking indicators 310, can be stored in one or more data structures in a memory of the computing device coupled to the tool 305. The information about the tool can be provided (e.g., via a bar code scanner, one or more communications signals, a wireless transmission, etc.) to a tracking computing device, such as the tool tracking system 705, or the image processing system 100, to perform the techniques described herein. The tool end 330 can be, for example, a drill bit, a biopsy needle, a cannula needle, or any other type of surgical tool end that can be positioned within a patient.

The tool 305 can include a communications line 335. The communications line 335 can include one or more wires, fiber-optic cables, or other data transmission lines capable of facilitating the transfer of information from the computing device of the tool 305 to another, external computing device (e.g., the tool tracking system 705, etc.). In some implementations, the communications line 335 can include one or more power transmission lines that can provide electrical power to the tool 305 or the components (e.g., the computing device, the indicators 310, the display 340, etc.) mounted on the tool 305. In some implementations, the communications line 335 can include only a power transmission line, and the data communications can proceed via a wireless communication interface communicatively coupled to the computing device. In some implementations, the communications line 335 can include separate power lines for each of the components and the tool 305 itself. For example, the tool 305 may be a drill having a predetermined voltage or current requirement. Likewise the components mounted to the tool 305 may have different power requirements. To accommodate such differences, the communications line 335 can include additional power lines that each carries electrical power having different voltages and currents that correspond to the devices to which they are connected. In some implementations, the tool 305 can include power distribution circuitry (e.g., step-up converters, step-down converters, AC-to-DC converters, etc.) that convert power to meet the requirements of the tool 305.

The tool 305 can include a display 340. The display can be a liquid crystal display (LCD), an organic LED (OLED) display, or any other type of portable display. The display can be coupled to the computing device of the tool 305, and can receive instructions to display one or more positioning instructions or configuration menus to a user (e.g., a surgeon, or another medical professional, etc.). In some implementations, the display can have a predetermined refresh rate that matches a data rate of the computing device of the tool 305. In some implementations, the display can display a user interface that provides prompts to a surgeon to move the tool 305 according to differences between current tool end 330 position and the target position within the patient. In some implementations, the display instructions are received via a wireless interface (e.g., Bluetooth, WiFi, NFC, etc.).

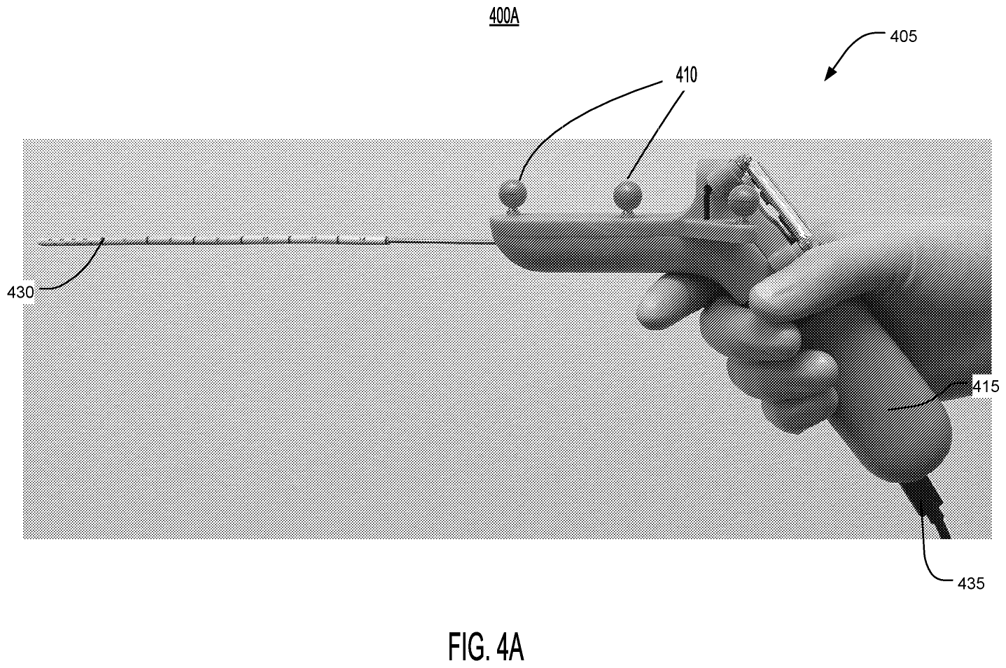

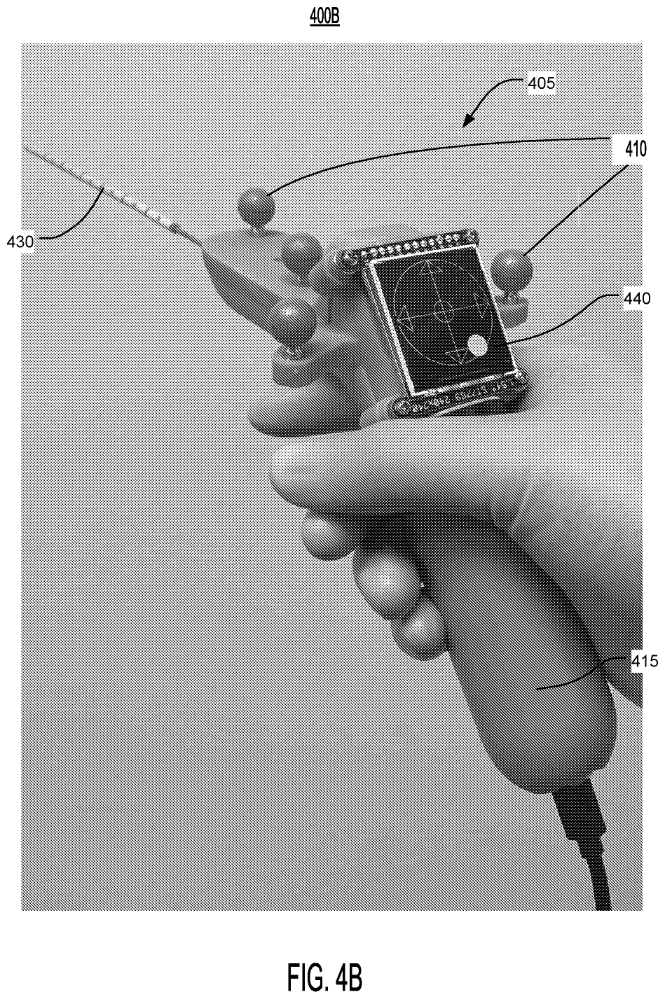

Referring now to FIGS. 4A and 4B, depicted are respective perspective views 400A and 400B of an implementation of a tool 405. The tool 405 can be similar to the tool 305 described herein above in conjunction with FIGS. 3A and 3B. The tool 405 can include one or more indicators 410, a grip portion 415, a tool end 430, a communications line 435, and a display 440. The tool 405, as depicted in FIGS. 4A and 4B, can be considered an example testing tool, and may not necessarily be used to perform any particular procedure on a patient. Nevertheless, the configuration of the tool 405 is such that it can be used for testing purposes for the computing device (e.g., the computing device described herein above in conjunction with FIG. 3, the tool computing system 705, etc.) of the tool 405.

The grip portion 415 can be similar to the grip portion 315 described herein above in conjunction with FIGS. 3A and 3B. As depicted in FIGS. 4A and 4B, the grip portion 415 can be shaped like a handle, or another sort of ergonomic shape to facilitate holding the tool 405 for long periods of time (e.g., during a surgical procedure, etc.). In some implementations, the grip portion 415 can be a housing for one or more components of the tool 405, such as a computing device (e.g., the tool computing system 720, the computing device described herein above in conjunction with FIGS. 3A and 3B, etc.).

As shown in FIGS. 4A and 4B, the tracking indicators 410 are ball tracking indicators. The ball tracking indicators can, in some implementations, be painted or colored such that they reflect specific wavelengths of light outside of the visual spectrum. Said wavelengths of light (e.g. IR light, etc.) can appear bright in images captured, for example, by the image capture devices 104. In some implementations, the tracking indicators 410 can be formed to have a specific shape that can be detected using one or more image processing algorithms. Although pictured here as having four tracking indictors 410, it should be understood that other numbers of indicators 410 can be used. For example, some tools could have more than four indicators 410, such as five, six, seven, eight, nine, or more indicators. In some implementations, the tool 405 can have at least three indicators 410.

Likewise, the tool end 430 can be similar to the tool end 330 described herein above in conjunction with FIGS. 3A and 3B. The tool end 430 depicted in FIGS. 4A and 4B is an example tool end, with length markings along its shaft. The tool end 430 may not necessarily be used in any surgical procedure, but may be used to calibrate or otherwise test the computing device integrated in the device. The communications line 435 can be similar to the communications line 335, and can facilitate the transmission of information between the computing device of the tool 405 and one or more external computing devices, such as the tool tracking system 705. In some implementations, the communications line 435 can provide power via one or more power transmission wires to the tool 405.

As shown in FIG. 4B, the display 440 can be similar to the display 340 described herein above in conjunction with FIGS. 3A and 3B. The display 440 can show a user interface that indicates to a user how to reposition the tool 405 such that the tool end 430 reaches a target location, for example, a biopsy location within a patient. As shown, the user interface displayed on the display 440 includes arrows that indicate directions, and a point that can be guided to the middle of the display 440 by changing the position (e.g., location, orientation, etc.) of the tool 405. The user interface can be displayed, for example, in response to receiving display instructions from an external computing system that tracks the position of the tool 405, such as the tool tracking system 705. The dot presented on the display 440 can also change in size to indicate that the tool 405 should be moved forward or backward to cause the tool end 430 to reach the target position.

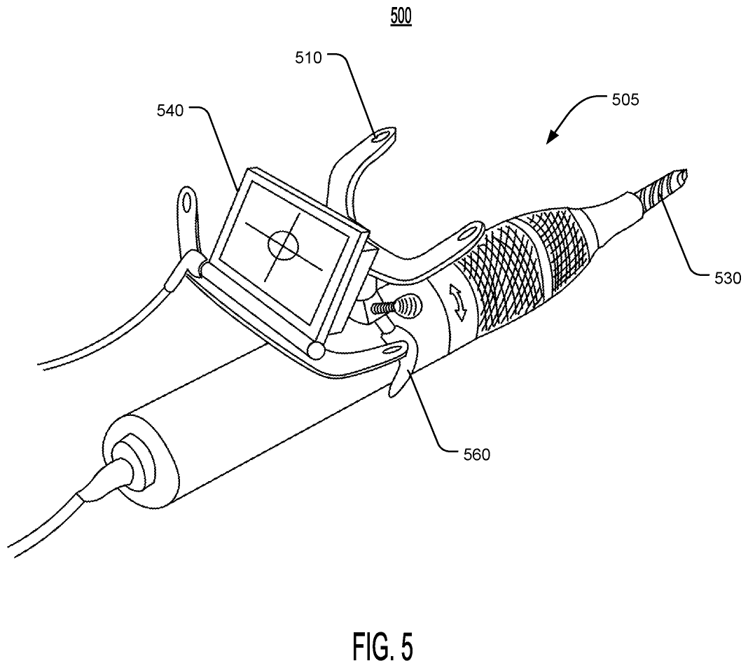

Referring now to FIG. 5, depicted is a perspective view 500 of an example tool 505 that is coupled to a bracket 560 that includes the components described herein above in conjunction with FIGS. 3A, 3B, 4A, and 4B. As shown in FIG. 5, the tool 505 can be coupled to an instrument attachment mechanism, such as a bracket 560. For example, the tool 505 can be a tool that would be otherwise expensive to manufacture with an integrated display (e.g., a specialized surgical drill, other specialized surgical equipment, etc.). In such circumstances, a traditional, or unmodified, surgical tool 505 can be coupled to the components described herein above in conjunction with FIGS. 3A, 3B, 4A, and 4B using a bracket 560. The bracket 560 can include structural elements, such as arms, that can hold and support components such as the display 540 and the tracking indicators 510. The display 540 can be similar to the displays 340 and the display 440 described herein above in conjunction with FIGS. 3A-4B, and each can perform any of the functionalities of the displays and tracking indicators as described herein.

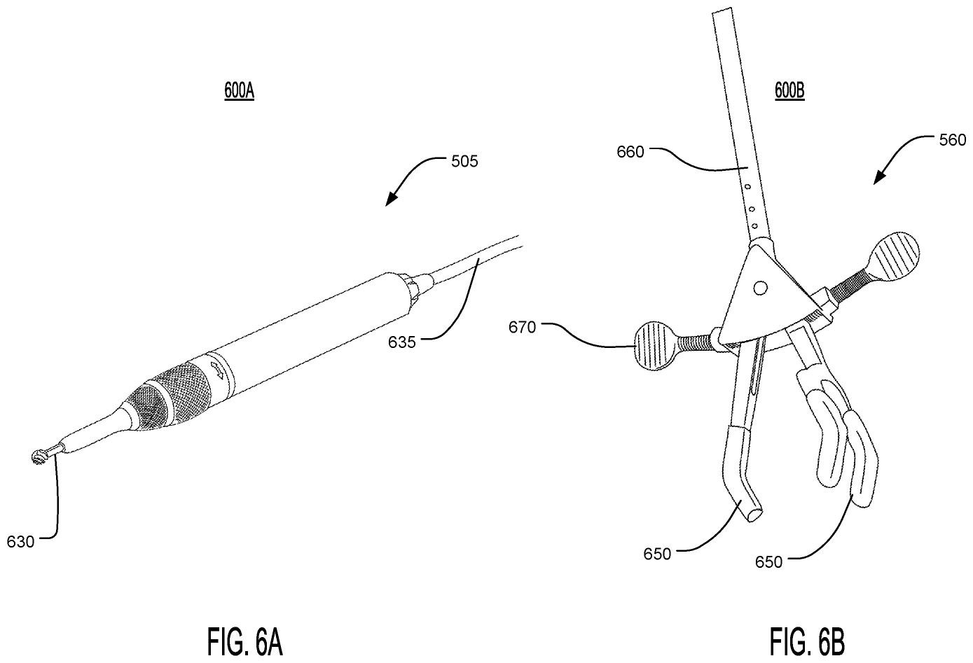

The bracket 560 can be, for example, a clamp that attaches to the housing of the tool 505, and is secured in place using one or more tightening bolts or clips. An example of a clamp-type bracket 560 is depicted in FIG. 6B. In some implementations, the bracket 560 can be attached via one or more threaded screw holes on the body of the tool 505. For example, the tool 505 may be configured to attach different accessories, or may be configured to be mounted onto another structure. Using screws, the bracket 560 can be secured to the housing of the tool 505. The bracket 560 assembly can include an embedded computing device, such as the tool computing device 720, or the computing devices described herein above in conjunction with FIGS. 3A-4B. The bracket 560 assembly can include its own communications line, similar to the communications lines 335 and 435 described herein above in conjunction with FIGS. 3A-3B, and 4A-4B respectively. The communications line of the bracket 560 assembly can provide power to any of the components coupled to the bracket 560 assembly, such as the display 540 and the tracking indicators 510.

In some implementations, the communications line of the bracket 560 assembly can be electrically coupled to, and receive power from, an electrical output of the tool 505. For example, the tool 505 may have one or more power interfaces that the communications line of the bracket 560 assembly can attach to and receive power from. The bracket 560 can include power distribution circuitry (e.g., DC-to-DC converters, AC-to-DC converters, etc.) that distribute appropriate amounts of voltage and current to each of the components coupled to the bracket 560. The communications line of the bracket 560 assembly can also be used, as described above, to communicate data or other information between the computing device mounted on the bracket 560 assembly and one or more external computing devices (e.g., the tool tracking system 705, etc.).

Referring now to FIG. 6A, depicted is a view 600A of the example tool 505 of FIG. 5 with the bracket 560 removed. The tool 605 can be, as shown in FIG. 6A, a surgical drilling tool. The drilling tool can include its own communications line 630, which can supply power to the drilling tool and, in some implementations, control signals that change the direction or speed of the drilling tool. The tool 505 can include its own tool end 630, which can be positioned at target locations within a patient to carry out surgical procedures. Although the tool 505 is shown in FIGS. 5 and 6A as being a power drill, it should be understood that different types of tools can be used in conjunction with the bracket 560 assembly shown in FIGS. 5 and 6B, such as biopsy needles, cannula needs, or power saws, among others.

Referring now to FIG. 6B, depicted is a view 600B of the example bracket 560 assembly shown in FIG. 5. The bracket 560 shown in FIG. 6B is depicted without any of the mounted components (e.g., the computing device, the screen 540, the tracking indicators 510, etc.). The bracket 560 shown is a clamp implementation of the bracket 560, in which arms 650 are used to clamp the bracket 560 in place prior to performing a calibration procedure. The clamps 650 can be secured, for example, using the tightening bolts 670. The components of the bracket 560 can be mounted on the shaft 660 using one or more threaded screws or other clamps. In some implementations, the components of the bracket 560 assembly can be positioned on one or more arms or structures that attach to the shaft of the bracket 560. Although a clamp-based bracket 560 is shown in FIG. 6B, it should be understood that other configurations, such as those attached using screws, bolts, or clip-on connectors, among others, are possible.

Referring now to FIG. 7, depicted is an example system 700 for tracking and providing display instructions to a tool (e.g., the tool 305, 405, 505, etc.) with an integrated display, in accordance with one or more implementations. The system 700 can include at least one tool tracking system 705 and at least one tool computing system 720. The tool tracking system 705 can include at least one tool information identifier 730, at least one tracking data receiver 735, at least one tool position determiner 740, at least one tool adjustment determiner 745, at least one tool instructions generator 760, and at least one tool communicator 765. The tool computing system 720 can include one or more indicators 710 and at least one display 750.

Each of the components (e.g., the tool tracking system 705, the tool computing system 720, etc.) of the system 700 can be implemented using the hardware components or a combination of software with the hardware components of a computing system (e.g., computing system 900, any other computing system described herein, etc.) detailed herein in conjunction with FIGS. 9A and 9B. Each of the components of the tool tracking system 705 (e.g., the tool information identifier 730, the tracking data receiver 735, the tool position determiner 740, the tool adjustment determiner 745, the tool instructions generator 760, the tool communicator 765, etc.) can perform the functionalities detailed herein.

The tool tracking system 705 can be, or form a part of, the image processing system 100 described herein in conjunction with FIGS. 1A, 1B, and 2, and can perform any of the functionalities of the image processing system 100 as described herein. The tool tracking system 705 can include at least one processor and a memory, e.g., a processing circuit. The memory can store processor-executable instructions that, when executed by processor, cause the processor to perform one or more of the operations described herein. The processor can include a microprocessor, an application-specific integrated circuit (ASIC), a field-programmable gate array (FPGA), etc., or combinations thereof. The memory can include, but is not limited to, electronic, optical, magnetic, or any other storage or transmission device capable of providing the processor with program instructions. The memory can further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor can read instructions. The instructions can include code from any suitable computer programming language. The tool tracking system 705 can include one or more computing devices or servers that can perform various functions as described herein. The tool tracking system 705 can include any or all of the components and perform any or all of the functions of the computer system 900 described herein in conjunction with FIGS. 9A and 9B.

The tool computing system 720 can be the computing system that is mounted on, or otherwise coupled to, a surgical tool such as the tools 305, 405, or 505 described herein in conjunction with FIGS. 3A-3B, 4A-4B, and 5, respectively, and can perform any of the functionalities of those computing devices as described herein. The tool computing system 720 can include at least one processor and a memory, e.g., a processing circuit. The memory can store processor-executable instructions that, when executed by processor, cause the processor to perform one or more of the operations described herein. The processor can include a microprocessor, an application-specific integrated circuit (ASIC), a field-programmable gate array (FPGA), etc., or combinations thereof. The memory can include, but is not limited to, electronic, optical, magnetic, or any other storage or transmission device capable of providing the processor with program instructions. The memory can further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor can read instructions. The instructions can include code from any suitable computer programming language. The tool computing system 720 can include one or more computing devices or servers that can perform various functions as described herein. The tool computing system 720 can include any or all of the components and perform any or all of the functions of the computer system 900 described herein in conjunction with FIGS. 9A and 9B.

The tool computing system 720 can include one or more indicators, which can be similar to the indicators 310, 410, or 510 described herein in conjunction with FIGS. 3A-3B, 4A-4B, and 5. The indicators 710 can include one or more LEDs, such as IR LEDs, or color LEDs that emit light in the visible spectrum. In some implementations, the tool computing system 720 can turn the indicators on and off, for example, in response to user input or in response to display instructions received from the too tracking system 705. The indicators 710 can include ball-shaped reflection devices, which reflect IR light and appear to flow when captured by an IR camera. Similar structures, other than ball-shaped structures are also possible. In some implementations, the indicators can be other types of markers, such as a QR code. The indicators 710 can be mounted on the tool to which the tool computing system 720 is coupled. In a bracket implementation, the indicators 710 can be mounted on one or more arms or supporting structures of the bracket, which can be coupled to a surgical tool as described here in.

The tool computing system 720 can include a display 750. The display 750 can be similar to the display 340, 440, and 540 described herein in conjunction with FIGS. 3A-3B, 4A-4B, and 5. The display 750 can be configured to present one or more user interfaces, for example, in accordance with display instructions received from the tool tracking system 705. The display 750 can be any kind of display, such as an LCD display, an LED display, or an OLED display, among others. In some implementations, the display 750 can display images streamed from the tool tracking system 705, such as renderings constructed from the images captured from the image capture devices 104. In some implementations, the display 750 can display some or all of the information displayed on the display 120 as described herein (e.g., portion a 3D rendering of a scene with indicators of target locations, etc.). The display 750 can be mounted on the tool to which the tool computing system 720 is coupled. In a bracket implementation, the indicators 710 can be mounted on one or more arms or supporting structures of the bracket, which can be coupled to a surgical tool as described herein.

Referring now to the functionality of the tool tracking system 705, the tool information identifier 730 can identify tool information from a tool to which the tool computing system 720 is mounted. The tool information can include a type of tool (e.g., a drill, needle, etc.), dimensions of the tool, such as width, length, and height, as well as the relative position of a tool end (e.g., the tool end 330, 430, or 530, etc.) to the indicators 710 positioned on the tool, and the relative positions of the indicators 710 to one another, among others. Identifying the tool information can include transmitting, via a network or another type of suitable communications interface (e.g., the communications lines 335, 435, or 535, etc.), a request for tool information to the tool computing system 720. In response, the tool computing system 720 can transmit a response including the requested tool information in one or more messages. Once received, the tool information identifier 730 can store the tool information in one or more data structures in the memory of the tool tracking system 705. For example, the tool information identifier 730 can store the tool information in association with information about a surgical procedure that will be performed. In some implementations, the tool information identifier 730 can receive a selection of a surgical procedure to be performed via one or more user interfaces provided by the tool tracking system 705. The user interfaces can be provided, for example, on the display 120 described herein above in conjunction with FIGS. 1A and 1B.

In some implementations, the tool information identifier 730 can retrieve information about a surgical procedure that will be performed using the tool to which the tool computing system 720 is coupled. For example, the tool information identifier 730 can retrieve information from memory that stores information about a patient that will be operated on. The patient, or the information about the patient, can be specified via input to a user interface presented on a display, such as the display 120. Upon receiving a specification of a patient, the tool information identifier 730 can retrieve one or more 3D images of the patient, which can be co-registered to a real-time 3D image of the patient captured using the image capture devices 104 described herein above in conjunction with FIGS. 1A-1B and 2. The 3D image of the patient can include a 3D point indicating a target location, which when mapped to the patient in a surgical environment, corresponds to a location on the patient that will be operated on. The 3D image can be a computed tomography (CT) scan image, a magnetic-resonance imaging (MRI) scan image, or any other type of 3D medical image of a patient. In some implementations, a 3D image of a patient can include more than one target location, and each target location can be associated with a target location identifier. Collectively, the target location identifiers can form a list of target location identifiers. In some implementations, target locations can be specified via input to a user interface, or from an internal configuration setting.

In some implementations, the tool information identifier 730 can perform one or more calibration procedures with the tool to which to the tool computing system 720 is coupled. Calibration procedures can be performed, for example, when the tool computing system 720 is coupled to a bracket (e.g., the bracket 560, etc.). In some implementations, the tool information identifier 730 can send one or more signals to the tool computing device 730 that cause the tool computing device to begin a calibration procedure. In some implementations, the tool information identifier 730 can send a signal that requests information about whether the tool computing system 720 is calibrated. In response, the tool computing system 720 can transmit a response message indicating whether the tool computing system 720 is calibrated (e.g., has stored, in computer memory, the relative positions of the indicators 710 and the tool end of the tool to which the tool computing device 720 is coupled, etc.). In some implementations, the tool information identifier 730 can maintain (e.g., store, etc.) the relative positions of the tool end to the indicators 710, and the tool information identifier 730 can determine whether the tool computing system 720 is calibrated by accessing one or more data structures stored in the memory of the tool tracking system 705.

If the tool computing system 720 is calibrated, the response message transmitted to the tool information identifier 730 can include the relative positions of the indicators 710 to one another and to the tool end. In some implementations, the relative positions of the indicators 710 to one another and to the tool end can be maintained by the tool information identifier 730 and retrieved from the memory of the tool tracking system 705, without requesting the information from the tool computing system 720. If the tool computing system 720 is not calibrated, the response message transmitted to the tool information identifier 730 can include an indication that the tool computing system 720 is not calibrated. If the tool information identifier 730 determines that the tool computing system 720 is not calibrated, the tool information identifier 730 can send instructions to the tool computing system 720 that cause the tool computing system 720 to present a calibration message on the display. The calibration message can include information about the tool computing system 720 that prompts the user to calibrate the tool computing system 720.

One type of calibration technique used by the tool information identifier 730 can be pivot calibration. Pivot calibration can include inserting the tool end of the tool to which the tool computing device is coupled into a cone. The tip of the tool end rests at the center (bottom) of the cone, and the user can rotate the tool, while the indicators 710 face the camera of the tool tracking system, such that the shaft of the tool end rotates about the inside surface of the cone. Because the cone dimensions (e.g., angle, depth, etc.) are known, the tool information identifier 730 can determine the relative position of the tool end to the indicators 710 by monitoring the position of the indicators 710 with respect to the position of the base of the cone. The position at the base of the cone can be treated as an additional special point and, as the indicators 710 are moved in response to moving the tool end about the surface of the cone, the relative position of the indicators 710 to the end of the tool end can be calculated.

To do so, the tool information identifier 730 can estimate the translation (e.g., relative change in position, etc.) from a dynamic reference frame defined by the positions of each of the indicators 710, as the dynamic reference frame is rotated about the code. The set of rigid transformations defined by the changing positions of the indicators can be represented as [Rᵢ, tᵢ]_{i=1 . . . m}, and the translation of the origin of the dynamic reference frame to the pivot (e.g., the tool end) can be represented as ^{DRF}t, and the translation from the tracker origin (e.g., the position of the image capture devices 104, etc.) to the tool end can be represented as ^{W}t. In general, the translation of the origin of the tracker to the tool end is useful, because it can be used to map the position of the tool end into the 3D scene captured by the image capture devices based on the detected positions of the indicators 710.

One example algorithm for estimating these translations is a sphere fitting algorithm, which relies on the observation that the locations of the dynamic reference frame (e.g., the translational component of the input transformations [Rᵢ, tᵢ]_{i=1 . . . m}, etc.) are all the surface of a sphere whose center is _{W}t. To estimate the sphere center a least squares formulation can be used, initially using an analytic estimate which minimizes an algebraic distance:

$${δ}_{i} = {( {t}_{i} {-}^{W} t )}^{T} ( {t}_{i} {-}^{W} t ) - {r}^{2}$$

Defining k=^{W}t^{TW}t−r², the following overdetermined equation system can be solved:

$$[ \begin{matrix}- 2 {t}_{1}^{T} & 1 \\ ⋮ & ⋮ \\ - 2 {t}_{m}^{T} & 1\end{matrix} ] [ \begin{matrix}{w}_{t} \\ k\end{matrix} ] = [ \begin{matrix}- {{t}_{1}}^{T} {t}_{1} \\ ⋮ \\ - {{t}_{1}}^{T} {t}_{1}\end{matrix} ] ,$$

which can then be refined using non-linear minimization, the Levenberg-Marquardt method, of the squared geometric distance:

$${δ}_{i}^{2} = {( \sqrt{{( {t}_{i} {-}^{W} t )}^{T} ( {t}_{i} {-}^{W} t )} - r )}^{2}$$

How that the value of ^{W}t is computed, the value of ^{DRF}t can be computed using the following equation:

$${DRF}^{} t = mean ( {R}_{i}^{T} {[}^{W} t - {t}_{i} ] )$$

Another example algorithm for estimating the translations ^{DRF}t and ^{W}t is an algebraic one-step method. The algebraic one-step method can be based on the observation that the tool end is pivoting around a fixed point, and therefore for all transformations, we have:

$${{R}_{i}}^{DRF} t + {t}_{i} {=}^{W} t$$

Thus, both translations can be estimated at once by solving the following overdetermined system of equations:

$$[ \begin{matrix}{R}_{1} & - I \\ ⋮ & ⋮ \\ {R}_{m} & - I\end{matrix} ] [ \begin{matrix}{DRF}^{} t \\ {W}^{} t\end{matrix} ] = [ \begin{matrix}- {t}_{1} \\ ⋮ \\ - {t}_{m}\end{matrix} ]$$

One other example algorithm for the estimating translations ^{DRF}t and ^{W}t is an algebraic two-step method. This method is based on the observation that the tool is pivoting around a fixed point, and therefore for any two transformations, we have:

$${R}_{i}^{DRF} t + {t}_{i} = {R}_{j}^{DRF} t + {t}_{j}$$

Thus, the values of ^{DRF}t can be estimated by solving the following overdetermined equation system:

$$[ \begin{matrix}{R}_{1} - {R}_{2} \\ ⋮ \\ {R}_{m - 1} - {R}_{m}\end{matrix} ] [ {DRF}^{} t ] = [ \begin{matrix}{t}_{2} - {t}_{1} \\ ⋮ \\ {t}_{m} - {t}_{m - 1}\end{matrix} ]$$

Using the computed value of ^{DRF}t, the value of ^{W}t can be computed using the following equation:

$${W}^{} t = mean ( {R}_{i}^{DRF} t + {t}_{i} )$$

Once this relative position (e.g., translation, etc.) is calculated, it can be stored in association with the tool type in one, and with a tool identifier, in one or more data structures in the memory of the tool tracking system 705. In some implementations, the tool computing system 720 can be calibrated by applying known amount of force to the tool end, and measuring the amount of displacement using a camera.

Once the target locations in the 3D medical images of the patient have been selected, and the tool computing system 720 has been calibrated, if needed, the tool can be used in a surgical environment to perform a surgical procedure on a patient. Prior to conducting the surgery, the tool tracking system 705 can co-register the 3D image of the patient with a real-time 3D image captured using the image capture devices 104, described herein above in conjunction with FIGS. 1A-1B and 2. The process of 3D image co-registration is described in great detail in International Application No. PCT/US2020/046473, the content of which is incorporated herein in its entirety. Co-registering the 3D medical image to the real-time image can superimpose the 3D medical image over the image of the patient in the reference frame of the image capture devices 104. A rendering of the co-registered environment can be rendered, for example, on the display 120 of the platform 112. In some implementations, one or more of the target locations can also be co-registered with the 3D scene captured by the image capture devices 104, and similarly rendered on the display 120 as a highlighted point or some other kind of indicator.

Responsive to the 3D image being co-registered with the real-time 3D image of the patient, the tracking data receiver 735 can capture tracking data from the indicators 710. As described herein above, the indicators 710 can be IR indicators that emit or reflect IR light that is captured by the IR sensors (e.g., or the image capture devices 104, etc.) of the image processing system 100 (which can be, or include, the tool tracking system 705). In some implementations, the tracking data receiver 735 can receive the positions of the points as three-dimensional points within the scene captured by the image capture devices 104 (e.g., if the image capture devices 104 are 3D cameras, etc.). The tracking data receiver 735 can receive the points in real-time, or when an image is captured by the image capture devices 104. Because the image capture devices 104 both construct the scene and capture the positions of the indicators 710, the 3D points that represent the positions of the indicators 710 in 3D space can be in the same reference frame as both the 3D scene and the co-registered 3D medical image.

Using the position data points received by the tracking data receiver 735, the tool position determiner 740 can determine the position (e.g., and orientation, etc.,) of the tool to which the indicators 710 are coupled. This can include retrieving the calibration information from the memory of the tool tracking system 705, and applying one or more transforms (e.g., translations, etc.) to the data points that represent the positions of the indicators 710 in the three-dimensional scene. The transformations can be translations, such as the values of ^{DRF}t and ^{W}t described herein above. In some implementations, if the relative position of the tool end and the indicators 710 is known (e.g., the tool is an integrated device, such as the tool depicted in FIGS. 3A and 3B, etc.), the transformations (e.g., translations, etc.) can be retrieved from one or more data structures associated with the tool. For example, the ^{W}t translation can be used to calculate the position of the tool end in the reference frame of the image capture devices 104 by transforming the detected positions of the indicators 710. The position of the tool end in the 3D can be estimated in real-time, for example, each time a frame is captured by the image capture devices 104. In some implementations, the position of the tool end in the 3D scene can be determined on a periodic basis, for example, five times per second. The estimated position of the tool end can be rendered (e.g., as an indicator point, or some other highlighted area, etc.) in the 3D scene on the display 120.

The tool adjustment determiner 745 can determine an amount by which the tool should be moved (or rotated, etc.) based on a difference between the estimated position of the tool end and a selected target location. As described herein, the display 750 of the tool computing device 720 can display an interface that allows a user to select a target location (e.g., one of the target locations specified as part of the surgical procedure, etc.). The selection can be a selection of a target location identifier, which the tool adjustment determiner 745 can use to retrieve the target location position information.

The tool adjustment determiner 745 can calculate the difference between the location of the tool end in the 3D scene and the selected target location in the 3D scene. The difference can be determined as a three-dimensional distance vector. For example, the tool adjustment determiner 745 can determine a Euclidean distance vector between the selected target location and the estimated location of the tool end. In some implementations, the difference can be determined in a coordinate space other than a Cartesian coordinate space, such as a cylindrical coordinate space or a spherical coordinate space. The difference vector between the target location and the estimated location of the tool end can calculated on a periodic basis, or when a new estimation of the position of the tool end is calculated as described above. The difference vector between the target location and the estimated location of the tool end can be stored in one or more data structures in the memory of the tool tracking system 705. In some implementations, the tool adjustment determiner 745 can receive sensor information, such as readings from one or more accelerometers, gyroscopes, or inertial measurement units, coupled to the tool computing system 720. Using these values, the tool adjustment determiner 745 can change (e.g., add to, subtract from, etc.) the distance vector to compensate for motion of the tool computing system 720.

The tool instructions generator 760 can generate display instructions for presentation on the display 750 of the tool computing device 720. The display instructions can include one or more prompts for the user to move the tool closer to the target location. In some implementations, the tool instructions generator 760 can determine a direction for prompting the user to move the tool to bring the tool end closer to the target location in the 3D scene. For example, the tool instructions generator 760 can transform the distance vector into a reference frame of the display 750 using the determined positions of the indicators 710. By transforming the difference vector into the reference frame of the display of the tool computing system 720, the tool instructions generator can compute the relative amounts by which the user should move the tool to bring the tool end closer to the target location. The reference frame of the display can have a first axis that is parallel to the tool end shaft of tool, and two other axes perpendicular to the first axis. The first axis can correspond to a depth dimension (e.g., an amount by which the tool is pushed forward or moved backward, etc.), and the other two axes can correspond to moving the tool upwards or downwards and left or right. By decomposing the distance vector inside the reference frame of the tool computing system 720, the tool instructions generator 760 can determine an amount by which the user should move the tool left/right, up/down, or forward/backward.

Once these directional changes have been determined, the tool instructions generator 760 can generate display instructions that correspond to the change in each direction. The display instructions can be instructions that cause the tool computing system 720 to present one or more arrows on the display 750. An example of such a user interface, which includes arrows, and a dot indicating the target location, is depicted in FIG. 4B. The display instructions can cause the tool computing system 720 to create a dot that represents the target location on screen, based on the direction which the tool should be moved. In some implementations, having the dot positioned at the center of the screen can indicate to a user that the tool is positioned properly in at least two axes (e.g., the up/down and left/right axes, etc.). Guiding arrows and lines can indicate the axes of the reference frame of the screen. In some implementations, the size of the dot can indicate the position of the tool end on the first axis (e.g., the forward/back axis). For example, the display instructions cause the dot to appear larger on the display 750 if the device should be moved backward to reach the target point. Furthering this example, the dot can appear smaller if the device should be moved forward long the axis. In some implementations, the display can show a ring in the center of the display, the size of which can indicate the position of the tool computing system 720. The display instructions can include instructions to display one or more indicators (e.g., a change in color, a message, etc.) when the tool end is positioned at the target location. The tool instructions generator 760 can also generate instructions that cause the tool computing system 720 to present one or more menus on the display 750. For example, the display instructions can show configuration menus (e.g., changing settings or preferences about the user interface, brightness settings, color settings, time settings, etc.).

The tool communicator 765 can communicate the display instructions to the tool computing system 720. As described herein above, the display instructions can include instructions that cause the display to show one or more user interfaces. The tool communicator 765 can transmit the display instructions, for example, via one or more communications lines, such as the communications lines 335 and 435 described herein above in conjunction with FIGS. 3A-3B and 4A-4B. In some implementations, the tool communicator 765 can communicate display instructions to the tool computing system 720 using a wireless communications interface (e.g., Bluetooth, WiFi, near-field communication, etc.). The tool communicator 765 can receive sensor information, for example, information from one or more accelerometers, gyroscopes, or other inertial measurement units, from sensors coupled to the tool computing system 720 for use in the operations described herein.

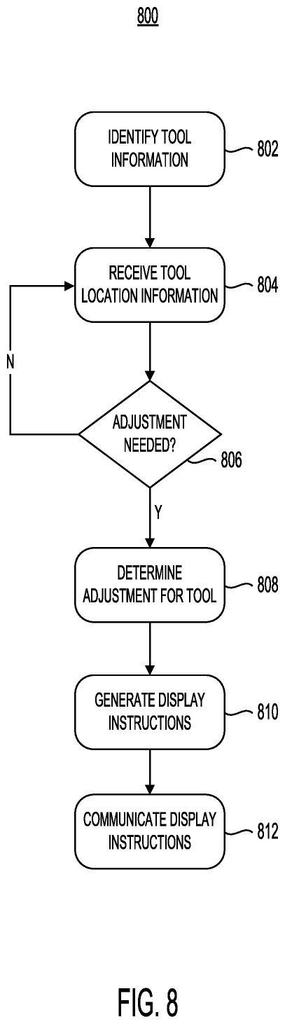

Referring now to FIG. 8, depicted is an example method 800 of tracking and providing display instructions to a tool with an integrated display, in accordance with one or more implementations. The method can be performed, for example, by the tool tracking system 705, or any other computing device described herein, including the computing system 900 described herein below in conjunction with FIGS. 9A and 9B. In brief overview, at STEP 802, the tool tracking system (e.g., the tool tracking system 705, etc.) can identify tool information. At step 804, the tool tracking system can receive tool location information. At STEP 806, the tool tracking system can determine whether an adjustment to the tool position is needed. At STEP 808, the tool tracking system can determine an adjustment for the tool. At STEP 810, the tool tracking system can generate display instructions. At STEP 812, the tool tracking system can communicate the display instructions. By performing these steps, the tool tracking system can provide accurate and timely guidance to a surgeon performing the surgical procedure. The tool tracking system does so by tracking a surgical tool used in the surgical procedure in real-time, and providing guidance prompts to the surgeon in real-time. Accuracy of the system is improved by dynamically mapping the location of the surgical tool to a reference frame of the patient, and by determining the relative translation or rotation of the surgical instrument needed for the surgical tool to reach a target location in the patient.

At STEP 802, the tool tracking system (e.g., the tool tracking system 705, etc.) can identify tool information. The tool information can include a type of tool (e.g., a drill, needle, etc.), dimensions of the tool, such as width, length, and height, as well as the relative position of a tool end (e.g., the tool end 330, 430, or 530, etc.) to the indicators (e.g., the indicators 710, etc.) positioned on the tool, and the relative positions of the indicators to one another, among others. Identifying the tool information can include transmitting, via a network or another type of suitable communications interface (e.g., the communications lines 335, 435, or 535, etc.), a request for tool information to a tool computing system (e.g., the tool computing system 720, etc.). In response, the tool computing system can transmit a response including the requested tool information in one or more messages. Once received, the tool tracking system can store the tool information in one or more data structures in the memory of the tool tracking system. For example, the tool tracking system can store the tool information in association with information about a surgical procedure that will be performed. In some implementations, the tool tracking system can receive a selection of a surgical procedure to be performed via one or more user interfaces provided by the tool tracking system. The user interfaces can be provided, for example, on the display 120 described herein above in conjunction with FIGS. 1A and 1B.

In some implementations, the tool tracking system can retrieve information about a surgical procedure that will be performed using the tool to which the tool computing system is coupled. For example, the tool tracking system can retrieve information from memory that stores information about a patient that will be operated on. The patient, or the information about the patient, can be specified via input to a user interface presented on a display, such as the display 120. Upon receiving such information, such as a specification of a patient, the tool tracking system can retrieve one or more 3D images of the patient, which can be co-registered to a real-time 3D image of the patient captured using the image capture devices 104 described herein above in conjunction with FIGS. 1A-1B and 2. The 3D image of the patient can include a 3D point indicating a target location, which when mapped to the patient in a surgical environment, corresponds to a location on the patient that will be operated on. The 3D image can be a computed tomography (CT) scan image, a magnetic-resonance imaging (MRI) scan image, or any other type of 3D medical image of a patient. In some implementations, a 3D image of a patient can include more than one target location, and each target location can be associated with a target location identifier. Collectively, the target location identifiers can form a list of target location identifiers. In some implementations, target locations can be specified via input to a user interface, or from an internal configuration setting.

In some implementations, the tool tracking system can perform one or more calibration procedures with the tool to which to the tool computing system is coupled. Calibration procedures can be performed, for example, when the tool computing system is coupled to a bracket (e.g., the bracket 560, etc.). In some implementations, an integrated tool, such as the tool 305 or 405 depicted in FIGS. 3A-3B and 4A-4B, respectively, may not require a calibration procedure, because the distance from the indicators to the tool end is determined during manufacturing, and stored in one or more data structures in the computing device of the tool. Therefore, in some implementations, the calibration information (e.g., the relative position of the tool end to the indicators, the relative positions of the indicators to one another, etc.) can be transmitted to the tool tracking system in response to one or more requests provided by the tool tracking system. In some implementations, the tool tracking system can send one or more signals to the tool computing device 730 that cause the tool computing device to begin a calibration procedure. In some implementations, the tool tracking system can send a signal that requests information about whether the tool computing system is calibrated. In response, the tool computing system can transmit a response message indicating whether the tool computing system is calibrated (e.g., has stored, in computer memory, the relative positions of the indicators and the tool end of the tool to which the tool computing device is coupled, etc.).

If the tool computing system is calibrated, the response message can include the relative positions of the indicators to one another and to the tool end. If the tool computing system is not calibrated, the response message can include an indication that the tool computing system is not calibrated. If the tool computing system is not calibrated, the tool tracking system can send instructions to the tool computing system that cause the tool computing system to present a calibration message on the display. The calibration message can include information about the tool computing system that prompts the user to calibrate the tool computing system.

One type of calibration technique used by the tool tracking system can be pivot calibration. Pivot calibration can include inserting the tool end of the tool to which the tool computing device is coupled into a cone. The tip of the tool end rests at the center (bottom) of the cone, and the user can rotate the tool, while the indicators face the camera of the tool tracking system, such that the shaft of the tool end rotates about the inside surface of the cone. Because the cone dimensions (e.g., angle, depth, etc.) are known, the tool tracking system can determine the relative position of the tool end to the indicators by monitoring the position of the indicators with respect to the position of the base of the cone. The position at the base of the cone can be treated as an additional special point and, as the indicators are moved in response to moving the tool end about the surface of the cone, the relative position of the indicators to the end of the tool end can be calculated.

To do so, the tool tracking system can estimate the translation (e.g., relative change in position, etc.) from a dynamic reference frame defined by the positions of each of the indicators, as the dynamic reference frame is rotated about the code. The set of rigid transformations defined by the changing positions of the indicators can be represented as [Rᵢ, tᵢ]_{i=1 . . . m}, and the translation of the origin of the dynamic reference frame to the pivot (e.g., the tool end) can be represented as ^{DRF}t, and the translation from the tracker origin (e.g., the position of the image capture devices 104, etc.) to the tool end can be represented as _{W}t. In general, the translation of the origin of the tracker to the tool end is useful, because it can be used to map the position of the tool end into the 3D scene captured by the image capture devices based on the detected positions of the indicators.

One example algorithm for estimating these translations is a sphere fitting algorithm, which relies on the observation that the locations of the dynamic reference frame (e.g., the translational component of the input transformations [Rᵢ, tᵢ]_{i=1 . . . m}, etc.) are all the surface of a sphere whose center is _{W}t. To estimate the sphere center a least squares formulation can be used, initially using an analytic estimate which minimizes an algebraic distance:

$${δ}_{i} = {( {t}_{i} {-}^{W} t )}^{T} ( {t}_{i} {-}^{W} t ) - {r}^{2}$$

Defining k=_{W}t^{TW}t−r², the following overdetermined equation system can be solved:

$$[ \begin{matrix}- 2 {t}_{1}^{T} & 1 \\ ⋮ & ⋮ \\ - 2 {t}_{m}^{T} & 1\end{matrix} ] [ \begin{matrix}{W}^{} t \\ k\end{matrix} ] = [ \begin{matrix}- {t}_{1}^{T} {t}_{1} \\ ⋮ \\ - {t}_{1}^{T} {t}_{1}\end{matrix} ] ,$$

which can then be refined using non-linear minimization, the Levenberg-Marquardt method, of the squared geometric distance:

$${δ}_{i}^{2} = {( \sqrt{{( {t}_{i} {-}^{W} t )}^{T} ( {t}_{i} {-}^{W} t )} - r )}^{2}$$

How that the value of ^{W}t is computed, the value of ^{DRF}t can be computed using the following equation:

$${DRF}^{} t = mean ( {R}_{i}^{T} {[}^{W} t - {t}_{i} ] )$$

Another example algorithm for estimating the translations ^{DRF}t and ^{W}t is an algebraic one-step method. The algebraic one-step method can be based on the observation that the tool end is pivoting around a fixed point, and therefore for all transformations, we have:

$${R}_{i}^{DRF} t + {t}_{i} {=}^{W} t$$

Thus, both translations can be estimated at once by solving the following overdetermined system of equations:

$$[ \begin{matrix}{R}_{1} & - I \\ ⋮ & ⋮ \\ {R}_{m} & - I\end{matrix} ] [ \begin{matrix}{DRF}^{} t \\ {W}^{} t\end{matrix} ] = [ \begin{matrix}- {t}_{1} \\ ⋮ \\ - {t}_{m}\end{matrix} ]$$

One other example algorithm for the estimating translations ^{DRF}t and ^{W}t is an algebraic two-step method. This method is based on the observation that the tool is pivoting around a fixed point, and therefore for any two transformations, we have:

$${R}_{i}^{DRF} t + {t}_{i} = {R}_{j}^{DRF} t + {t}_{j}$$

Thus, the values of ^{DRF}t can be estimated by solving the following overdetermined equation system:

$$[ \begin{matrix}{R}_{1} - {R}_{2} \\ ⋮ \\ {R}_{m - 1} - {R}_{m}\end{matrix} ] [ {DRF}^{} t ] = [ \begin{matrix}{t}_{2} - {t}_{1} \\ ⋮ \\ {t}_{m} - {t}_{m - 1}\end{matrix} ]$$

Using the computed value of ^{DRF}t, the value of ^{W}t can be computed using the following equation:

$${W}^{} t = mean ( {{R}_{i}}^{DRF} t - {t}_{i} )$$

Once this relative position (e.g., translation, etc.) is calculated, it can be stored in association with the tool type in one, and with a tool identifier, in one or more data structures in the memory of the tool tracking system. In some implementations, the tool computing system can be calibrated by applying known amount of force to the tool end, and measuring the amount of displacement using a camera.

At step 804, the tool tracking system can receive tool location information. As described herein above, the indicators coupled to the tool can be IR indicators that emit or reflect IR light that is captured by the IR sensors (e.g., or the image capture devices 104, etc.) of the image processing system 100 (which can be, or include, the tool tracking system). In some implementations, the tool tracking system can receive the positions of the points as three-dimensional points within the scene captured by the image capture devices (e.g., if the image capture devices are 3D cameras, etc.). The tool computing system 720 can receive the points in real-time, or when an image is captured by the image capture devices. Because the image capture devices both construct the scene and capture the positions of the indicators, the 3D points that represent the positions of the indicators in 3D space can be in the same reference frame as both the 3D scene and the co-registered 3D medical image.

At STEP 806, the tool tracking system can determine whether an adjustment to the tool position is needed, such as if an adjustment modification condition is satisfied. To do so, the tool tracking system can estimate the position of the tool end and compare it to a position of the selected target location. If the position of the tool end is within a predetermined threshold distance from the target location, then the tool tracking system can execute STEP 804. If the position of the tool end is not within a predetermined distance of the selected target location, the tool tracking system can execute STEP 808. The predetermined distance can be any distance at which the surgical tool can perform its intended function at, or relatively close to, the target patient. Some example distances include, for example, a centimeter, half a centimeter, or a millimeter, among other distances. The threshold distance may depend on the type of surgical procedure being performed. Using the position data points received from the tool computing system, the tool tracking system can determine the position (e.g., and orientation, etc.,) of the tool to which the indicators are coupled. This can include retrieving the calibration information from the memory of the tool tracking system, and applying one or more transforms (e.g., translations, etc.) to the data points that represent the positions of the indicators in the three-dimensional scene. The transformations can be translations, such as the values of ^{DRF}t and ^{W}t described herein above. In some implementations, if the relative position of the tool end and the indicators is known (e.g., the tool is an integrated device, such as the tool depicted in FIGS. 3A and 3B, etc.), the transformations (e.g., translations, etc.) can be retrieved from one or more data structures associated with the tool. For example, the ^{W}t translation can be used to calculate the position of the tool end in the reference frame of the image capture devices by transforming the detected positions of the indicators. The position of the tool end in the 3D can be estimated in real-time, for example, each time a frame is captured by the image capture devices 104.

In some implementations, the position of the tool end, and therefore the steps in the 3D scene can be determined on a periodic basis, for example, five times per second. However, it should be understood that the steps of the method 800 can be performed at any particular frequency to achieve desired results. For example, certain surgical procedures, or surgical tools, may require increased accuracy when performing position detection. In some implementations, to improve the accuracy of positon detection, the position of the tool end can be determined iteratively, and averaged over time (e.g., a rolling average over a predetermined number of position samples). The position determination procedure may be performed as a function of the display rate of the display positioned on the surgical tool. For example, if the display rate of the tool has a refresh rate of 20 Hz, the position of the tool end may be determined at a rate of 60 Hz, or three times per screen refresh. Each of the three samples may be averaged, and used as the estimated position of the tool end for that display cycle. Estimating the position of the tool end multiple times per refresh cycle can improve the overall accuracy of the instructions prompted to the surgeon using the techniques described herein. The estimated position of the tool end can be rendered (e.g., as an indicator point, or some other highlighted area, etc.) in the 3D scene on the display 120 of the image processing system 100.

At STEP 808, the tool tracking system can determine an adjustment for the tool. The tool tracking system can determine an amount by which the tool should be moved (or rotated, etc.) based on a difference between the estimated position of the tool end and a selected target location. As described herein, the display of the tool computing device can present an interface that allows a user to select a target location (e.g., one of the target locations specified as part of the surgical procedure, etc.). The selection can be a selection of a target location identifier, which the tool tracking system can use to retrieve the target location position information.

The tool tracking system can calculate the difference between the location of the tool end in the 3D scene and the selected target location in the 3D scene. The difference can be determined as a three-dimensional distance vector. For example, the tool tracking system can determine a Euclidean distance vector between the selected target location and the estimated location of the tool end. In some implementations, the difference can be determined in a coordinate space other than a Cartesian coordinate space, such as a cylindrical coordinate space or a spherical coordinate space. The difference vector between the target location and the estimated location of the tool end can calculated on a periodic basis, or when a new estimation of the position of the tool end is calculated as described above. The difference vector between the target location and the estimated location of the tool end can be stored in one or more data structures in the memory of the tool tracking system. In some implementations, the tool tracking system can receive sensor information, such as readings from one or more accelerometers, gyroscopes, or inertial measurement units, coupled to the tool computing system. Using these values, the tool tracking system can change (e.g., add to, subtract from, etc.) the distance vector to compensate for motion of the tool computing system.

At STEP 810, the tool tracking system can generate display instructions. The display instructions can include one or more prompts for the user to move the tool closer to the target location. In some implementations, the tool tracking system can determine a direction for prompting the user to move the tool to bring the tool end closer to the target location in the 3D scene. For example, the tool tracking system can transform the distance vector into a reference frame of the display mounted on the tool using the determined positions of the indicators. By transforming the difference vector into the reference frame of the display of the tool computing system, the tool instructions generator can compute the relative amounts by which the user should move the tool to bring the tool end closer to the target location. The reference frame of the display can have a first axis that is parallel to the tool end shaft of tool, and two other axes perpendicular to the first axis. The first access can correspond to a depth dimension (e.g., an amount by which the tool is pushed forward or moved backward, etc.), and the other two axes can correspond to moving the tool upwards or downwards and left or right. By decomposing the distance vector inside the reference frame of the tool computing system, the tool tracking system can determine an amount by which the user should move the tool left/right, up/down, or forward/backward.

Once these directional changes have been determined, the tool tracking system can generate display instructions that correspond to the change in each direction. The display instructions can be instructions that cause the tool computing system to present one or more arrows on the display of the tool computing system. An example of such a user interface, which includes arrows, and a dot indicating the target location, is depicted in FIG. 4B. The display instructions can cause the tool computing system to create a dot that represents the target location on screen, based on the direction which the tool should be moved. In some implementations, having the dot positioned at the center of the screen can indicate to a user that the tool is positioned properly in at least two axes (e.g., the up/down and left/right axes, etc.). Guiding arrows and lines can indicate the axes of the reference frame of the screen. In some implementations, the size of the dot can indicate the position of the tool end on the first axis (e.g., the forward/back axis). For example, the display instructions cause the dot to appear larger on the display if the device should be moved backward to reach the target point. Furthering this example, the dot can appear smaller if the device should be moved forward long the axis. In some implementations, the display can show a ring in the center of the display, the size of which can indicate the position of the tool computing system. The display instructions can include instructions to display one or more indicators (e.g., a change in color, a message, etc.) when the tool end is positioned at the target location. The tool tracking system can also generate instructions that cause the tool computing system to present one or more menus on the display. For example, the display instructions can show configuration menus (e.g., changing settings or preferences about the user interface, brightness settings, color settings, time settings, etc.).

At STEP 812, the tool tracking system can communicate the display instructions. As described herein above, the display instructions can include instructions that cause the display to show one or more user interfaces. The tool tracking system can transmit the display instructions, for example, via one or more communications lines, such as the communications lines 335 and 435 described herein above in conjunction with FIGS. 3A-3B and 4A-4B. In some implementations, the tool tracking system can communicate display instructions to the tool computing system using a wireless communications interface (e.g., Bluetooth, WiFi, near-field communication, etc.). The tool tracking system can receive sensor information, for example, information from one or more accelerometers, gyroscopes, or other inertial measurement units, from sensors coupled to the tool computing system for use in the operations described herein.

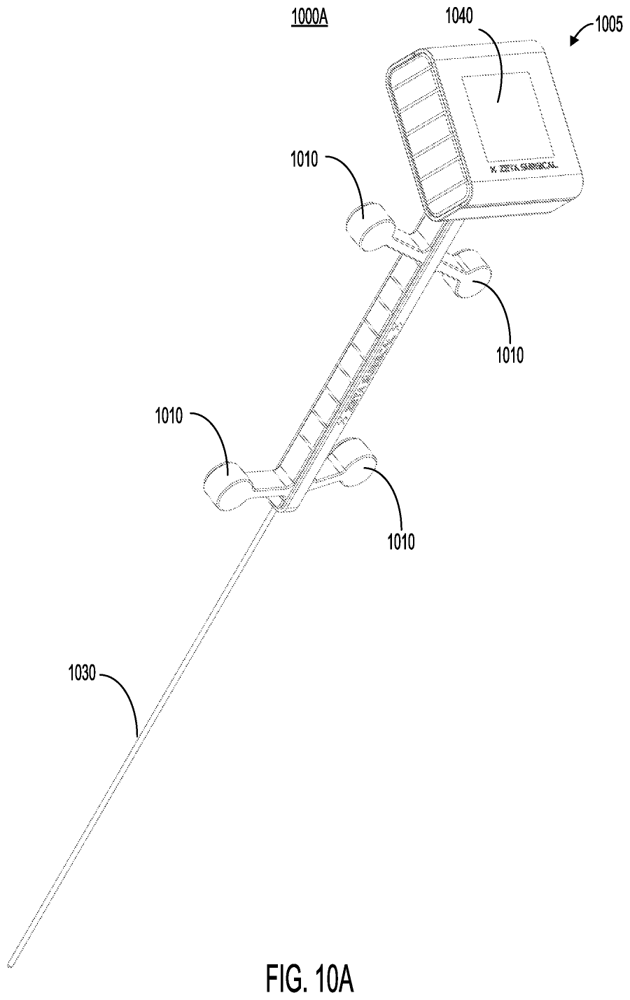

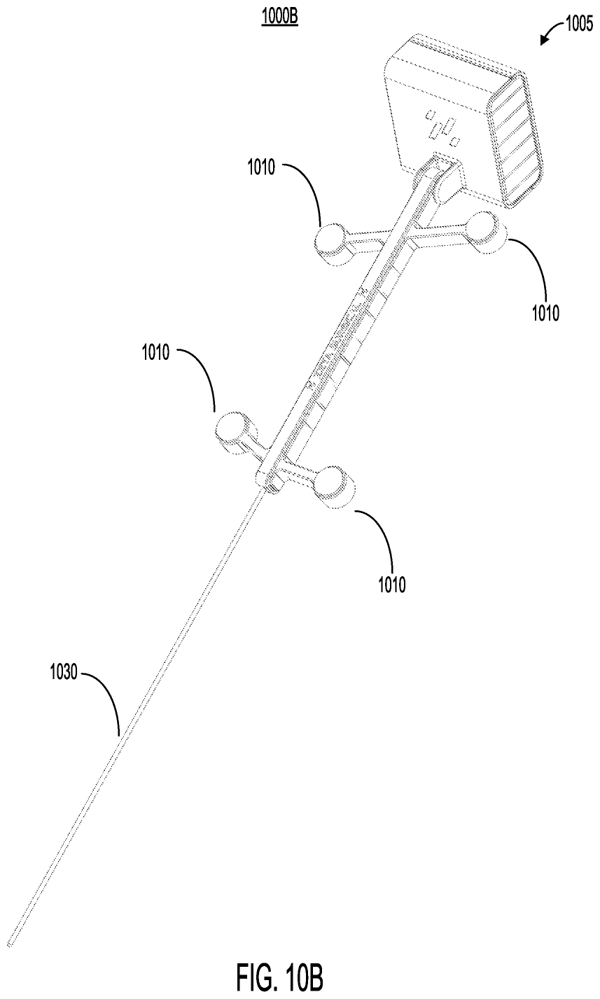

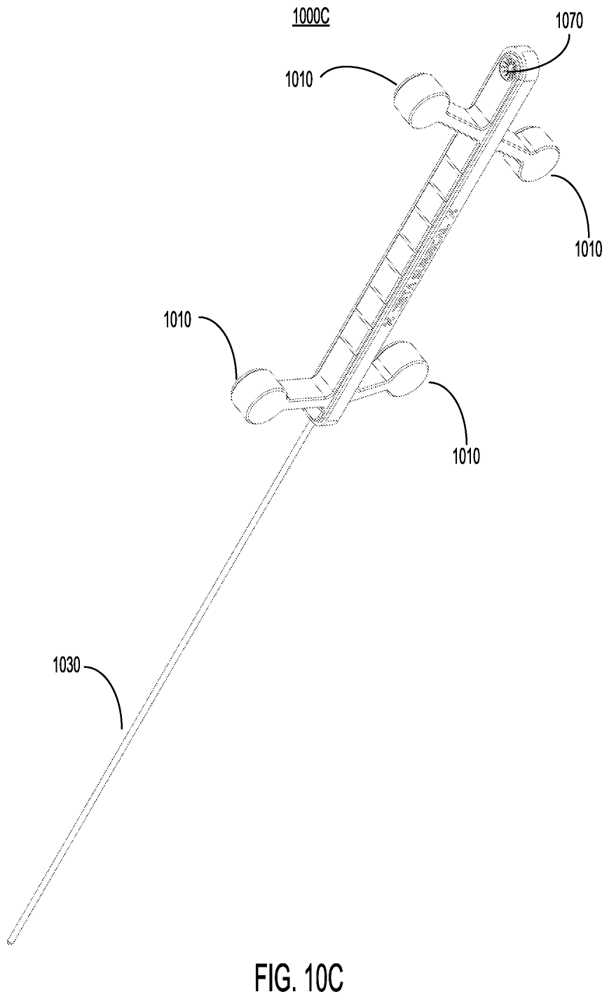

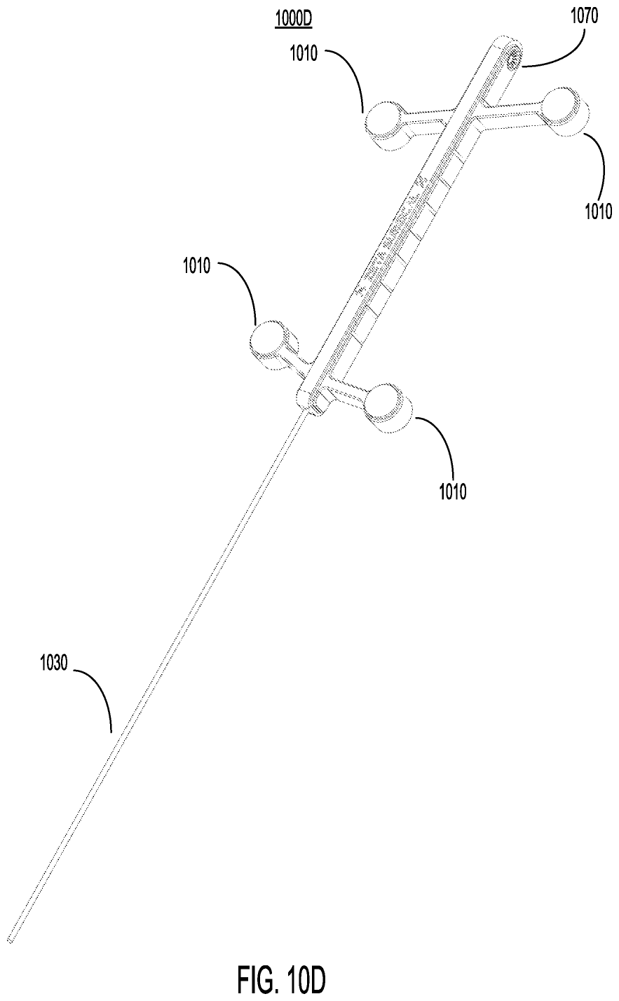

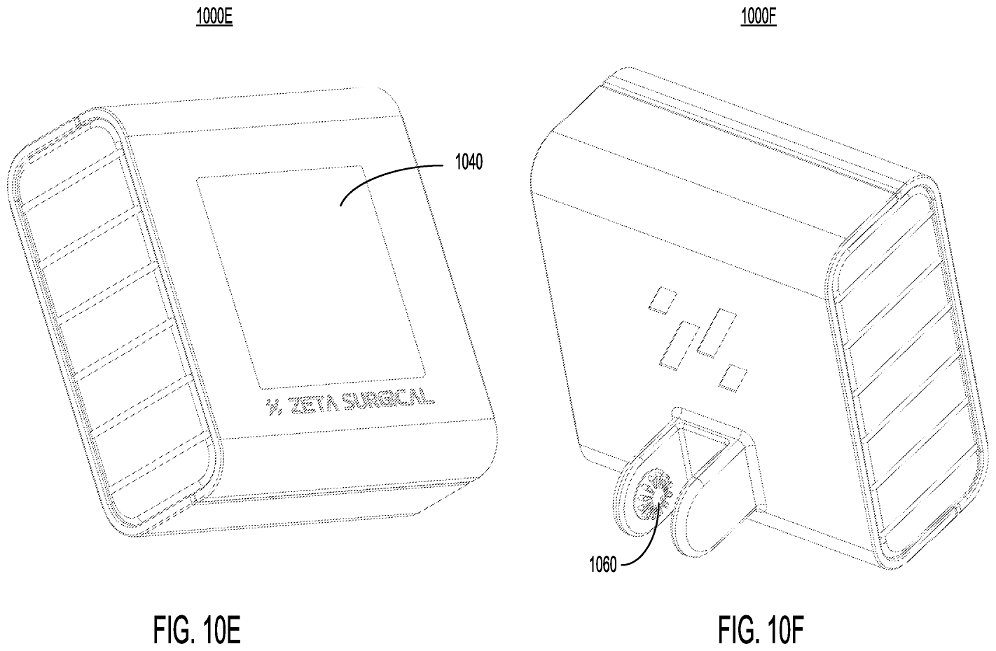

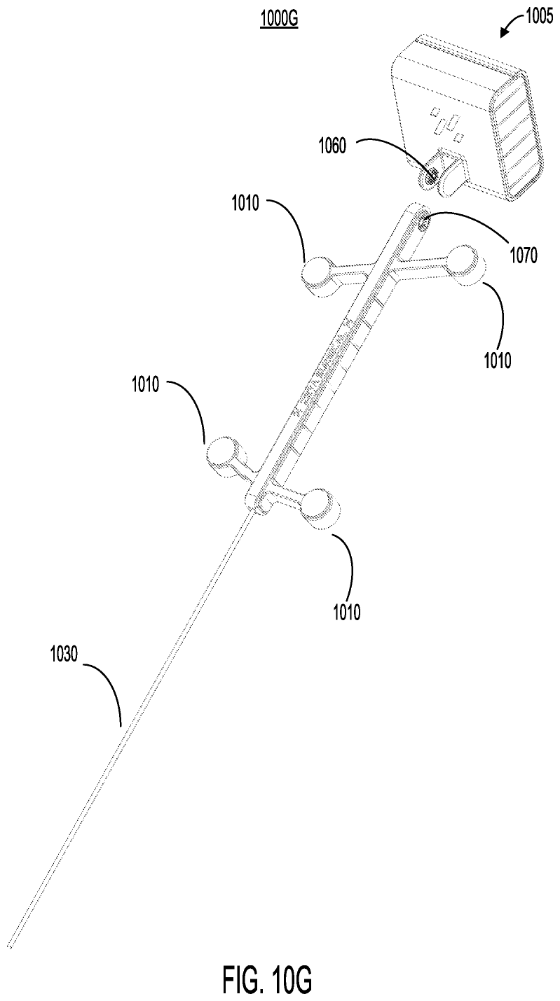

Referring to FIGS. 10A, 10B, 10C, 10D, 10E, 10F, and 10G, illustrated are example views of an example tool assembly 1005 similar to the devices described herein. Referring to FIG. 10A, illustrated is an example perspective view 1000A of the tool assembly 1005. The tool assembly 1005 can be similar to, and include any of the structure and functionality of, the surgical tool 305 described in connection with FIGS. 3A and 3B, the tool 405 as described in connection with FIGS. 4A and 4B, or the tool 505 described in connection with FIG. 5. As shown, the tool assembly 1005 includes can a tool end 1030, which can be positioned within the patient. The tool assembly 1005 may include a communications interface (not shown) that allows the tool assembly 1005 to communicate with a tracking computing device, such as the tool tracking system 705, or the image processing system 100, to perform the techniques described herein. The communications interface may be a wired or wireless communications interface, and may include power distribution circuitry as described herein.

The tool assembly 1005 may be held by a surgeon, robot, or other medical professional. The tool end 1030, which can have a tip portion, can be positioned by the surgeon at a target position within the patient to carry out a portion of a medical procedure, such as a biopsy. Information about the tool end 1030, such as the dimensions of the tool end, the type of tool end 1030, or the distance and orientation of the tool end 1030 from the tracking indicators 1010, can be stored in one or more data structures in a memory of the computing device communicatively coupled to the tool assembly 1005. The information about the tool can be provided (e.g., via a bar code scanner, one or more communications signals, a wireless transmission, etc.) to a tracking computing device, such as the tool tracking system 705 or the image processing system 100, to perform the techniques described herein. The information about the tool may indicate a relative length of the tool end 1030 from the indicators positioned on the tool. When used in a corresponding calibration procedure, this can improve the overall accuracy of the tool tracking techniques described herein. The tool end 1030 can be, for example, a drill bit, a biopsy needle, a cannula needle, or any other type of surgical tool end that can be positioned within a patient.

The tool assembly 1005 can include a display 1040. The display 1040 can be an LCD, an OLED display, or any other type of portable display. The display 1040 can be coupled to the computing device of the tool assembly 1005, which may be positioned within the housing of the display 1040, and can receive instructions to display one or more positioning instructions or configuration menus to a user (e.g., a surgeon, or another medical professional, etc.). In some implementations, the display can have a predetermined refresh rate that matches a data rate of the computing device in communication with the computing device of the tool assembly 1005. In some implementations, the display 1040 can display a user interface that provides guidance prompts to a surgeon to move the tool 1005 according to differences between the current tool end 1030 position and the target position within the patient. In some implementations, the display instructions are received via a wireless interface (e.g., Bluetooth, WiFi, NFC, etc.) or a wired interface.

The tool assembly 1005 can include tracking indicators 1010. The tracking indicators can be, for example, IR LEDs, LEDs that emit color in the visual spectrum, tracking balls colored with a predetermined color or having a predetermined, detectable shape, or other tracking features, such as QR codes. The tracking indicators 1010 can be positioned on predetermined places on the tool assembly 1005, and can form a matrix or array of sensors that, when detected by a computing device (e.g., the image processing system 100, the tool tracking system 705, etc.), can be used to determine a position and orientation of the tool assembly 1005. In some implementations, the tool assembly 1005 can include one or more position sensors, such as accelerometers, gyroscopes, or IMUs, among others.

Referring to FIG. 10B, illustrated is another example perspective view 1000B of the tool assembly 1005 shown in FIG. 10A. As shown, the display 1040 can include its own housing, which is coupled to the portion of the device having the tool end 1030 using a connector 1060. The connector may be a moveable connector, which allows the display 1040 to rotate around one or more axes when coupled to the portion of the tool assembly 1005 having the tool end 1040. Although the portion of the tool assembly 1005 having the tool end 1030 is shown as physically coupled to the indicators 1010, it should be understood that other configurations are also possible. For example, the indicators 1010 may be coupled to, or formed as a part of, the housing for the display 1040, which may form a separable portion of the tool assembly 1005.

Referring to FIGS. 10C and 10D, illustrated are perspective views 1000C and 1000D of a portion of the tool assembly 1005 described in connection with FIGS. 10A and 10B. As shown, the tool assembly 1005 may be separable from the display 1040, which may include its own housing and connector 1060, as described herein. The body of the tool assembly 1005, as shown, may include its own connector portion 1070 that receives, or otherwise couples to, the connector 1060 of the housing of the display 1040. In some implementations, the connector portion 1070 can include one or more communications signals or one or more power lines, either to receive energy to power components of the body of the tool assembly 1005 (e.g., the indicators 1010, the tool end 1030, etc.), or to provide energy to power the display 1040. In some implementations, the body of the tool assembly 1005 can include power distribution circuitry that converts electrical power received from an external source (not shown) to power the display 1040.

Referring to FIGS. 10E and 10F, illustrated are perspective views 1000E and 1000F of the display 1040. As shown, the display 1040 can include a housing, which may include a computing device that can perform the functionality of the other tool devices described herein. The display 1040 may include one or more power sources, such as batteries, and may include one or more communications interfaces, as described herein. Although not shown in this example, the housing for the display 1040 may include one or more of the indicators 1040. As shown, the housing of the display 1040 includes a connector 1060, which may couple to a corresponding connector 1070 on the body of the tool assembly 1005, as described in connection with FIGS. 10C and 10D. FIG. 10G shows a perspective view 1000G of the devices described in connection with FIGS. 10A-10F. As shown in the perspective view 1000G, the body of the tool assembly 1005 and the housing of the display 1040 have been separated from one another.

### B. Computing Environment

FIGS. 9A and 9B depict block diagrams of a computing device 900. As shown in FIGS. 9A and 9B, each computing device 900 includes a central processing unit 921, and a main memory unit 922. As shown in FIG. 9A, a computing device 900 can include a storage device 928, an installation device 916, a network interface 918, an I/O controller 923, display devices 924a-924n, a keyboard 926 and a pointing device 927, e.g. a mouse. The storage device 928 can include, without limitation, an operating system, software, and software of the image processing system 100 or the tool tracking system 700. As shown in FIG. 9B, each computing device 900 can also include additional optional elements, e.g. a memory port 903, a bridge 970, one or more input/output devices 930a-930n (generally referred to using reference numeral 930), and a cache memory 940 in communication with the central processing unit 921.

The central processing unit 921 is any logic circuitry that responds to and processes instructions fetched from the main memory unit 922. In many embodiments, the central processing unit 921 is provided by a microprocessor unit, e.g.: those manufactured by Intel Corporation of Mountain View, California; those manufactured by Motorola Corporation of Schaumburg, Illinois; the ARM processor (from, e.g., ARM Holdings and manufactured by ST, TI, ATMEL, etc.) and TEGRA system on a chip (SoC) manufactured by Nvidia of Santa Clara, California; the POWER7 processor, those manufactured by International Business Machines of White Plains, New York; or those manufactured by Advanced Micro Devices of Sunnyvale, California; or field programmable gate arrays (“FPGAs”) from Altera in San Jose, CA, Intel Corporation, Xlinix in San Jose, CA, or MicroSemi in Aliso Viejo, CA, etc. The computing device 900 can be based on any of these processors, or any other processor capable of operating as described herein. The central processing unit 921 can utilize instruction level parallelism, thread level parallelism, different levels of cache, and multi-core processors. A multi-core processor can include two or more processing units on a single computing component. Examples of multi-core processors include the AMD PHENOM IIX2, INTEL CORE i5 and INTEL CORE i7.

Main memory unit 922 can include one or more memory chips capable of storing data and allowing any storage location to be directly accessed by the microprocessor 921. Main memory unit 922 can be volatile and faster than storage 928 memory. Main memory units 922 can be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 922 or the storage 928 can be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 922 can be based on any of the above described memory chips, or any other available memory chips capable of operating as described herein. In the embodiment shown in FIG. 9A, the processor 921 communicates with main memory 922 via a system bus 950 (described in more detail below). FIG. 9B depicts an embodiment of a computing device 900 in which the processor communicates directly with main memory 922 via a memory port 903. For example, in FIG. 9B the main memory 922 can be DRDRAM.

FIG. 9B depicts an embodiment in which the main processor 921 communicates directly with cache memory 940 via a secondary bus, sometimes referred to as a backside bus. In other embodiments, the main processor 921 communicates with cache memory 940 using the system bus 950. Cache memory 940 typically has a faster response time than main memory 922 and is typically provided by SRAM, BSRAM, or EDRAM. In the embodiment shown in FIG. 9B, the processor 921 communicates with various I/O devices 930 via a local system bus 950. Various buses can be used to connect the central processing unit 921 to any of the I/O devices 930, including a PCI bus, a PCI-X bus, or a PCI-Express bus, or a NuBus. For embodiments in which the I/O device is a video display 924, the processor 921 can use an Advanced Graphics Port (AGP) to communicate with the display 924 or the I/O controller 923 for the display 924. FIG. 9B depicts an embodiment of a computer 900 in which the main processor 921 communicates directly with I/O device 930b or other processors 921′ via HYPERTRANSPORT, RAPIDIO, or INFINIBAND communications technology. FIG. 9B also depicts an embodiment in which local busses and direct communication are mixed: the processor 921 communicates with I/O device 930a using a local interconnect bus while communicating with I/O device 930b directly.

A wide variety of I/O devices 930a-930n can be present in the computing device 900. Input devices can include keyboards, mice, trackpads, trackballs, touchpads, touch mice, multi-touch touchpads and touch mice, microphones (analog or MEMS), multi-array microphones, drawing tablets, cameras, single-lens reflex camera (SLR), digital SLR (DSLR), CMOS sensors, CCDs, accelerometers, inertial measurement units, infrared optical sensors, pressure sensors, magnetometer sensors, angular rate sensors, depth sensors, proximity sensors, ambient light sensors, gyroscopic sensors, or other sensors. Output devices can include video displays, graphical displays, speakers, headphones, inkjet printers, laser printers, and 3D printers.

Devices 930a-930n can include a combination of multiple input or output devices, including, e.g., Microsoft KINECT, Nintendo Wiimote for the WII, Nintendo WII U GAMEPAD, or Apple IPHONE. Some devices 930a-930n allow gesture recognition inputs through combining some of the inputs and outputs. Some devices 930a-930n provides for facial recognition which can be utilized as an input for different purposes including authentication and other commands. Some devices 930a-930n provides for voice recognition and inputs, including, e.g., Microsoft KINECT, SIRI for IPHONE by Apple, Google Now or Google Voice Search.

Additional devices 930a-930n have both input and output capabilities, including, e.g., haptic feedback devices, touchscreen displays, or multi-touch displays. Touchscreen, multi-touch displays, touchpads, touch mice, or other touch sensing devices can use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices can allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, can have larger surfaces, such as on a table-top or on a wall, and can also interact with other electronic devices. Some I/O devices 930a-930n, display devices 924a-924n or group of devices can be augmented reality devices. The I/O devices can be controlled by an I/O controller 921 as shown in FIG. 9A. The I/O controller 921 can control one or more I/O devices, such as, e.g., a keyboard 126 and a pointing device 927, e.g., a mouse or optical pen. Furthermore, an I/O device can also provide storage and/or an installation medium 116 for the computing device 900. In still other embodiments, the computing device 900 can provide USB connections (not shown) to receive handheld USB storage devices. In further embodiments, an I/O device 930 can be a bridge between the system bus 950 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 924a-924n can be connected to I/O controller 921. Display devices can include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays can use, e.g. stereoscopy, polarization filters, active shutters, or autostereoscopy. Display devices 924a-924n can also be a head-mounted display (HMD). In some embodiments, display devices 924a-924n or the corresponding I/O controllers 923 can be controlled through or have hardware support for OPENGL or DIRECTX API or other graphics libraries.

In some embodiments, the computing device 900 can include or connect to multiple display devices 924a-924n, which each can be of the same or different type and/or form. As such, any of the I/O devices 930a-930n and/or the I/O controller 923 can include any type and/or form of suitable hardware, software, or combination of hardware and software to support, enable or provide for the connection and use of multiple display devices 924a-924n by the computing device 900. For example, the computing device 900 can include any type and/or form of video adapter, video card, driver, and/or library to interface, communicate, connect or otherwise use the display devices 924a-924n. In one embodiment, a video adapter can include multiple connectors to interface to multiple display devices 924a-924n. In other embodiments, the computing device 900 can include multiple video adapters, with each video adapter connected to one or more of the display devices 924a-924n. In some embodiments, any portion of the operating system of the computing device 900 can be configured for using multiple displays 924a-924n. In other embodiments, one or more of the display devices 924a-924n can be provided by one or more other computing devices 900a or 900b connected to the computing device 900, via the network 940. In some embodiments software can be designed and constructed to use another computer's display device as a second display device 924a for the computing device 900. For example, in one embodiment, an Apple iPad can connect to a computing device 900 and use the display of the device 900 as an additional display screen that can be used as an extended desktop. One ordinarily skilled in the art will recognize and appreciate the various ways and embodiments that a computing device 900 can be configured to have multiple display devices 924a-924n.

Referring again to FIG. 9A, the computing device 900 can comprise a storage device 928 (e.g. one or more hard disk drives or redundant arrays of independent disks) for storing an operating system or other related software, and for storing application software programs such as any program related to the software for the image processing system 100 or the tool tracking system 700. Examples of storage device 928 include, e.g., hard disk drive (HDD); optical drive including CD drive, DVD drive, or BLU-RAY drive; solid-state drive (SSD); USB flash drive; or any other device suitable for storing data. Some storage devices can include multiple volatile and non-volatile memories, including, e.g., solid state hybrid drives that combine hard disks with solid state cache. Some storage device 928 can be non-volatile, mutable, or read-only. Some storage device 928 can be internal and connect to the computing device 900 via a bus 950. Some storage device 928 can be external and connect to the computing device 900 via an I/O device 930 that provides an external bus. Some storage device 928 can connect to the computing device 900 via the network interface 918 over a network, including, e.g., the Remote Disk for MACBOOK AIR by Apple. Some client devices 900 may not require a non-volatile storage device 928 and can be thin clients or zero clients 202. Some storage device 928 can also be used as an installation device 916, and can be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Computing device 900 can also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc.

Furthermore, the computing device 900 can include a network interface 918 to interface to the network 940 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 900 communicates with other computing devices 900′ via any type and/or form of gateway or tunneling protocol e.g. Secure Socket Layer (SSL) or Transport Layer Security (TLS), or the Citrix Gateway Protocol manufactured by Citrix Systems, Inc. of Ft. Lauderdale, Florida. The network interface 918 can comprise a built-in network adapter, network interface card, PCMCIA network card, EXPRESSCARD network card, card bus network adapter, wireless network adapter, USB network adapter, modem or any other device suitable for interfacing the computing device 900 to any type of network capable of communication and performing the operations described herein.

A computing device 900 of the sort depicted in FIG. 9A can operate under the control of an operating system, which controls scheduling of tasks and access to system resources. The computing device 900 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 7000, WINDOWS Server 2012, WINDOWS CE, WINDOWS Phone, WINDOWS XP, WINDOWS VISTA, and WINDOWS 7, WINDOWS RT, and WINDOWS 8 all of which are manufactured by Microsoft Corporation of Redmond, Washington; MAC OS and iOS, manufactured by Apple, Inc. of Cupertino, California; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, California, among others. Some operating systems, including, e.g., the CHROME OS by Google, can be used on zero clients or thin clients, including, e.g., CHROMEBOOKS.

The computer system 900 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 900 has sufficient processor power and memory capacity to perform the operations described herein. In some embodiments, the computing device 900 can have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 900 is a gaming system. For example, the computer system 900 can comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, or an XBOX 360 device manufactured by the Microsoft Corporation of Redmond, Washington, or an OCULUS RIFT or OCULUS VR device manufactured BY OCULUS VR, LLC of Menlo Park, California.

In some embodiments, the computing device 900 is a digital audio player such as the Apple IPOD, IPOD Touch, and IPOD NANO lines of devices, manufactured by Apple Computer of Cupertino, California. Some digital audio players can have other functionality, including, e.g., a gaming system or any functionality made available by an application from a digital application distribution platform. For example, the IPOD Touch can access the Apple App Store. In some embodiments, the computing device 900 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the computing device 900 is a tablet e.g. the IPAD line of devices by Apple; GALAXY TAB family of devices by Samsung; or KINDLE FIRE, by Amazon.com, Inc. of Seattle, Washington. In other embodiments, the computing device 900 is an eBook reader, e.g. the KINDLE family of devices by Amazon.com, or NOOK family of devices by Barnes & Noble, Inc. of New York City, New York.

In some embodiments, the communications device 900 includes a combination of devices, e.g. a smartphone combined with a digital audio player or portable media player. For example, one of these embodiments is a smartphone, e.g. the IPHONE family of smartphones manufactured by Apple, Inc.; a Samsung GALAXY family of smartphones manufactured by Samsung, Inc.; or a Motorola DROID family of smartphones. In yet another embodiment, the communications device 900 is a laptop or desktop computer equipped with a web browser and a microphone and speaker system, e.g. a telephony headset. In these embodiments, the communications devices 900 are web-enabled and can receive and initiate phone calls. In some embodiments, a laptop or desktop computer is also equipped with a webcam or other video capture device that enables video chat and video call.

In some embodiments, the status of one or more machines 900 in the network are monitored, generally as part of network management. In one of these embodiments, the status of a machine can include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information can be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

Implementations of the subject matter and the operations described in this specification can be implemented in digital electronic circuitry, or in computer software embodied on a tangible medium, firmware, or hardware, including the structures disclosed in this specification and their structural equivalents, or in combinations of one or more of them. Implementations of the subject matter described in this specification can be implemented as one or more computer programs, e.g., one or more components of computer program instructions, encoded on computer storage medium for execution by, or to control the operation of, data processing apparatus. The program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can include a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The terms “data processing apparatus”, “data processing system”, “client device”, “computing platform”, “computing device”, or “device” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program can, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatuses can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The elements of a computer include a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), for example. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube), plasma, or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can include any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

While this specification contains many specific implementation details, these should not be construed as limitations on the scope of any inventions or of what can be claimed, but rather as descriptions of features specific to particular implementations of the systems and methods described herein. Certain features that are described in this specification in the context of separate implementations can also be implemented in combination in a single implementation. Conversely, various features that are described in the context of a single implementation can also be implemented in multiple implementations separately or in any suitable subcombination. Moreover, although features can be described above as acting in certain combinations and even initially claimed as such, one or more features from a claimed combination can in some cases be excised from the combination, and the claimed combination can be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In some cases, the actions recited in the claims can be performed in a different order and still achieve desirable results. In addition, the processes depicted in the accompanying figures do not necessarily require the particular order shown, or sequential order, to achieve desirable results.

In certain circumstances, multitasking and parallel processing can be advantageous. Moreover, the separation of various system components in the implementations described above should not be understood as requiring such separation in all implementations, and it should be understood that the described program components and systems can generally be integrated together in a single software product or packaged into multiple software products.

Having now described some illustrative implementations and implementations, it is apparent that the foregoing is illustrative and not limiting, having been presented by way of example. In particular, although many of the examples presented herein involve specific combinations of method acts or system elements, those acts and those elements can be combined in other ways to accomplish the same objectives. Acts, elements and features discussed only in connection with one implementation are not intended to be excluded from a similar role in other implementations or implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular can also embrace implementations including a plurality of these elements, and any references in plural to any implementation or element or act herein can also embrace implementations including only a single element. References in the singular or plural form are not intended to limit the presently disclosed systems or methods, their components, acts, or elements to single or plural configurations. References to any act or element being based on any information, act or element can include implementations where the act or element is based at least in part on any information, act, or element.

Any implementation disclosed herein can be combined with any other implementation, and references to “an implementation,” “some implementations,” “an alternate implementation,” “various implementation,” “one implementation” or the like are not necessarily mutually exclusive and are intended to indicate that a particular feature, structure, or characteristic described in connection with the implementation can be included in at least one implementation. Such terms as used herein are not necessarily all referring to the same implementation. Any implementation can be combined with any other implementation, inclusively or exclusively, in any manner consistent with the aspects and implementations disclosed herein.

References to “or” can be construed as inclusive so that any terms described using “or” can indicate any of a single, more than one, and all of the described terms.

Where technical features in the drawings, detailed description or any claim are followed by reference signs, the reference signs have been included for the sole purpose of increasing the intelligibility of the drawings, detailed description, and claims. Accordingly, neither the reference signs nor their absence have any limiting effect on the scope of any claim elements.

The systems and methods described herein can be embodied in other specific forms without departing from the characteristics thereof. Although the examples provided can be useful transforming a three-dimensional point cloud to a different reference frame, the systems and methods described herein can be applied to other environments. The foregoing implementations are illustrative rather than limiting of the described systems and methods. The scope of the systems and methods described herein can thus be indicated by the appended claims, rather than the foregoing description, and changes that come within the meaning and range of equivalency of the claims are embraced therein.

1. A device, comprising:
a tool portion configured to be inserted into a target location in a patient during a procedure;
a display assembly mounted on the device and comprising a display, the display configured to display prompts for an operator of the device to guide the tool portion to the target location in the patient; and
a computing device having one or more processors coupled to memory, the computing device mounted on the device and configured to:
provide tool information to a controller computing device;
receive, from the controller computing device, instructions to present a change in a position of the device to bring the tool portion closer to the target location in the patient; and
present, based on the instructions, a guidance prompt for the operator that indicates the change in the position of the device.

2. The device of claim 1, wherein the device further comprises a grip portion that allows the operator to hold and position the device.

3. The device of claim 1, further comprising a housing that houses both the display assembly and the computing device.

4. The device of claim 3, wherein the tool portion is coupled to the housing.

5. The device of claim 1, further comprising a button that, when actuated, causes the tool portion to perform a function of the tool portion.

6. The device of claim 1, further comprising one or more position sensors, and wherein the computing device is further configured to receive the tool information from the one or more position sensors.

7. The device of claim 1, further comprising one or more indicators, each of the one or more indicators mounted at a respective predetermined position on the device.

8. The device of claim 1, wherein the tool portion is one of a catheter device, a drill device, a biopsy needle, or a cannula needle.

9. The device of claim 1, wherein the device comprises a respective power interface for each of the computing device and the tool portion.

10. The device of claim 1, further comprising a wireless interface, and wherein the computing device configured to provide the tool information and receive the instructions via the wireless interface.

11. A system, comprising:
a connector configured to couple to a body of a surgical tool;
a display assembly coupled to the connector and comprising a display, the display configured to display prompts for an operator of the surgical tool to guide the surgical tool to a target location in a patient; and
a computing device coupled to the display assembly or the bracket, the computing device comprising one or more processors coupled to memory, the computing device configured to:
provide tool information about the surgical tool to a controller computing device;
receive, from the controller computing device, instructions to present a change in a position of the surgical tool to bring the surgical tool closer to the target location in the patient; and
present, based on the instructions, a guidance prompt for the operator that indicates the change in the position of the surgical tool.

12. The system of claim 11, further comprising the surgical tool, wherein the surgical tool further comprises a grip portion that allows the operator to hold and position the surgical tool while the bracket is coupled to the body of the surgical tool.

13. The system of claim 11, wherein the connector comprises a clamp that couples to the body of the surgical tool.

14. The system of claim 11, wherein the connector is a bracket, and the display assembly or the computing device are coupled to the bracket using threaded screws or bolts.

15. (canceled)

16. The system of claim 11, further comprising one or more position sensors, and wherein the computing device is further configured to receive the tool information from the one or more position sensors.

17. The system of claim 11, further comprising one or more indicators, each of the one or more indicators mounted at a respective predetermined position on the system.

18. The system of claim 11, further comprising a communications interface via which the computing device communicates data with the controller computing device.

19. The system of claim 18, wherein the communications interface attaches to a power interface of the surgical tool to receive power for the computing device and the display assembly.

20. The system of claim 11, further comprising a wireless interface, and wherein the computing device configured to provide the tool information and receive the instructions via the wireless interface.

21. A method, comprising:
(a) identifying, by one or more processors coupled to memory, tool information from a tool having a mounted display assembly coupled to a computing device;
tracking, by the one or more processors, using signals received from an image capture device, a position of the tool based on determined positions of indicators mounted on the tool;
determining, by the one or more processors, a position of the tool in a three-dimensional (3D) reference frame that includes a target location in a patient;
determining, by the one or more processors, a change in the position of the tool that causes a portion of the tool to move closer to the target location in the 3D reference frame;
generating, by the one or more processors, based on the change in the position of the tool determined by the one or more processors, display instructions that cause the tool to display a prompt to a user of the tool to adjust the position of the tool; and
providing, by the one or more processors, the display instructions to the computing device mounted on the tool.

22-40. (canceled)
